(19) United States
(12) Patent Application Publication
(10) Pub. No.: US 2021/0368850 A1
(43) Pub. Date: Dec. 2, 2021

(54) VAPORIZABLE FORMULATION

(71) Applicant: NICOVENTURES TRADING LIMITED, London (GB)

(72) Inventors: Klaus Mathie, London (GB); Maria Montserrat Sanchez Pena, London (GB)

(21) Appl. No.: 17/051,490

(22) PCT Filed: May 3, 2019
(86) PCT No.: PCT/GB2019/051244

§ 371 Date: Oct. 29, 2020

(30) Foreign Application Priority Data
May 3, 2018 (GB) 1807305.6

(51) Int. Cl. A24B 15/167; A24B 15/34; A24B 15/32; A24B 15/38; A24B 15/40

(52) U.S. Cl. CPC A24B 15/167; A24B 15/34; A24B 15/406; A24B 15/38; A24B 15/32

(57) ABSTRACT

There is provided a vaporizable formulation comprising (i) one or more solvents; and (ii) less than about 1 wt % of a cooling agent based on the weight of the vaporizable formulation; wherein the cooling agent volatilizes at a higher temperature than menthol at atmospheric pressure. There is also provided a vaporizable formulation comprising (i) one or more solvents; and (ii) less than about 12 wt % of a cooling agent based on the weight of the vaporizable formulation; wherein the cooling agent volatilizes at a higher temperature than menthol at atmospheric pressure and is a compound of formula (I) or a salt and/or solvate thereof as defined herein.

### PRIORITY CLAIM

The present application is a National Phase entry of PCT Application No. PCT/GB2019/051244, filed May 3, 2019, which claims priority from GB Patent Application No. 1807305.6, filed May 3, 2018, each of which is hereby fully incorporated herein by reference.

### FIELD OF THE INVENTION

The present disclosure relates to a vaporizable formulation, to containers in which are contained the vaporizable formulation and to electronic vapor provision systems such as electronic vapor delivery systems (e.g. e-cigarettes) incorporating said formulation.

### BACKGROUND TO THE INVENTION

Electronic vapor provision systems such as e-cigarettes generally contain a reservoir of liquid which is to be vaporized, typically containing nicotine. When a user inhales on the device, a heater is activated to vaporize a small amount of liquid, which is therefore inhaled by the user. The liquid for e-cigarettes may also contain a flavor component such as menthol to provide a sensory experience to the user. In some circumstances, the liquid may contain a flavor component without nicotine.

Menthol is, however, extremely volatile and vaporizes readily in the presence of heat. This high volatility causes problems for current e-cigarette devices since it can lead to degradation of flavor quality and consumer satisfaction. The volatility of menthol is also detrimental to the shelf-life of the liquid in or for the e-cigarette device.

### SUMMARY OF THE INVENTION

In a first characteristic there is provided a vaporizable formulation comprising (i) one or more solvents; and (ii) less than about 1 wt % of a cooling agent, based on the weight of the vaporizable formulation, wherein the cooling agent volatilizes at a higher temperature than menthol at atmospheric pressure.

In a second characteristic there is provided a vaporizable formulation comprising (i) one or more solvents; and (ii) less than about 12 wt % of a cooling agent, preferably equal to or less than about 10 wt %, based on the weight of the vaporizable formulation, wherein the cooling agent volatizes at a higher temperature than menthol at atmospheric pressure and is a compound of formula (I), or a salt and/or solvate thereof:

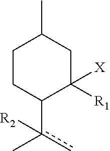

wherein X is hydrogen or OR′, wherein R′ is an alkyl group or an alkenyl group which may be taken together with R₁ to form a three to five-membered heterocyclyl group, wherein the heterocyclyl group is optionally substituted by one or more substituents selected from OH, O-alkyl, alkyl-OH, alkyl-O-alkyl, NH₂, NH-alkyl, N-(alkyl)₂, NO₂ and CN; and wherein R₁ and R₂ are each independently selected from hydrogen, OH, ORₐ, C(O)NR_{b}R_{c} and C(O)OR_{b}R_{c}; with the proviso that when R₁ is OH the compound of formula (I) is not menthol; and when the double bond is present, R₂ is absent;

wherein Rₐ is an alkyl group, an alkenyl group, a C(O)R_{f} group, or a C(O)-alkyl-C(O)R_{f} group wherein the alkyl groups and alkenyl groups are optionally substituted by one or more substituents selected from OH, O-alkyl, NH₂, NH-alkyl, N-(alkyl)₂, NO₂ and CN; and wherein R_{f} is an alkyl group, an alkenyl group, OH, O-alkyl, NH₂, NH-alkyl or N-(alkyl)₂, wherein the alkyl groups and alkenyl groups are optionally substituted by one or more substituents selected from OH, O-alkyl, NH₂, NH-alkyl, N-(alkyl)₂, NO₂ and CN;

wherein R_{b} and R_{c} are each independently hydrogen, an alkyl group, an alkenyl group, an aryl group, an aralkyl group, a heteroaryl group, or a heteroaralkyl group, wherein the alkyl groups, alkenyl groups, aryl groups and heteroaryl groups are optionally substituted by one or more substituents selected from OH, O-alkyl, NH₂, NH-alkyl, N-(alkyl)₂, NO₂, CN and C(O)R_{f}.

Unless specified otherwise, the detailed description below applies to both the first characteristic and the second characteristic of the invention.

The present invention further provides a process for forming a vapor, the process comprising (a) providing a vaporizable formulation as defined herein, and (b) vaporizing the formulation.

The present invention further provides a contained vaporizable formulation comprising (a) a container, and (b) a vaporizable formulation as defined herein.

The present invention further provides an electronic vapor provision system comprising (a) a vaporizer for vaporizing liquid for inhalation by a user of the electronic vapor provision system, (b) a power supply comprising a cell or battery for supplying power to the vaporizer, and (c) a vaporizable formulation as defined herein.

The present invention further provides use of a cooling agent for extending the shelf-life of a vaporizable formulation, wherein the cooling agent volatilizes at a higher temperature than menthol at atmospheric pressure.

### BRIEF DESCRIPTION OF THE DRAWINGS

The present invention will now be described in further detail by way of example only with reference to the accompanying figures in which:

FIGS. 1A to 1D are the thermogravimetric analysis traces for (1S,2R,5S)-N-ethyl-5-methyl-2-(propan-2-yl)cyclohexanecarboxamide (WS-3) in air (A and B) and in nitrogen (C and D).

FIGS. 2A to 2D are the thermogravimetric analysis traces for ethyl-2-[[(1R,2S,5R)-5-methyl-2-propan-2-ylcyclohexanecarbonyl]amino] acetate (WS-5) in air (A and B) and in nitrogen (C and D).

FIGS. 3A to 3D are the thermogravimetric analysis traces for (1R,2S,5R)-N-(4-methoxyphenyl-p-menthanecarboxamide (WS-12) in air (A and B) and in nitrogen (C and D).

FIGS. 4A to 4D are the thermogravimetric analysis traces for N,2,3-trimethyl-2-propan-2-ylbutanamide (WS-23) in air (A and B) and in nitrogen (C and D).

FIGS. 5A to 5D are the thermogravimetric analysis traces for (1R,2S,5R)-N-(2-(pyridin-2-yl)ethyl)menthylcarboxamide (Evercool® 190) in air (A and B) and in nitrogen (C and D).

FIGS. 6A to 6D are the thermogravimetric analysis traces for (−)-menthone 1,2-glycerol ketal (Frescolat® MGA) in air (A and B) and in nitrogen (C and D).

FIGS. 7A to 7D are the thermogravimetric analysis traces for (−)-menthyl lactate (Frescolat® ML) in air (A and B) and in nitrogen (C and D).

FIGS. 8A to 8D are the thermogravimetric analysis traces for 3-((−)-menthoxy)propane-1,2-diol (Coolact® 10) in air (A and B) and in nitrogen (C and D).

FIGS. 9A to 9D are the thermogravimetric analysis traces for (−)-menthyl succinate (monomenthyl succinate) in air (A and B) and in nitrogen (C and D).

FIG. 10 is a scatter plot of Tₚₑₐₖ mass loss (° C.) against molecular weight (g mol⁻¹) for the compounds shown below in Table 1 and menthol.

FIG. 11 shows the results from the subjective cooling intensity testing carried out as described in Example 1 below.

FIG. 12 shows the results from the cooling intensity testing carried out as described in Example 2 below.

### DETAILED DESCRIPTION

As discussed herein the present invention provides a vaporizable formulation comprising (i) one or more solvents, and either (ii) less than about 1 wt % of a cooling agent based on the weight of the vaporizable formulation, wherein the cooling agent volatilizes at a higher temperature than menthol at atmospheric pressure, or less than about 12 wt % of a cooling agent based on the weight of the vaporizable formulation, wherein the cooling agent volatizes at a higher temperature than menthol at atmospheric pressure and is a compound of formula (I) or a salt and/or solvent thereof as defined above and herein.

The use of sensate compounds and particularly cooling agents is well documented in the food and pharmaceutical industry. Cooling agents are typically small organic molecules like menthol which deliver a cooling sensation to a user upon contact with the oral cavity, nasal cavity and/or skin. This cooling sensation falls under the category of chemesthetic sensations and arises because the small organic molecule activates certain receptors in the skin and/or mucous membranes. The experience of a cooling sensation thus relies on chemesthesis of the user. Chemesthesis is also referred to in the art as the “common chemical sense” or trigeminal chemosensation because it typically refers to sensations that are mediated by the trigeminal nerve and which are elements of the somatosensory system, distinguishing them from olfaction and taste.

Menthol is a widely used cooling agent. It is, for example, used in mouthwashes and topical analgesic creams to provide a cooling effect. This cooling effect is believed to result from the user's cold-sensitive TRPM8 receptors in the oral cavity, nasal cavity and/or skin being chemically triggered by menthol. A similar effect is seen when menthol is used as a tobacco additive. The menthol provides a minty odor and flavor when inhaled by the user.

Menthol is, however, problematic when included in a liquid for an e-cigarette device because it has the potential to cause respiratory irritation and is associated with a bitter taste and burning sensation when used at higher concentrations. Menthol is also an extremely volatile compound. It has a high vapor pressure at room temperature and a low boiling point at atmospheric pressure. At 25° C., for example, menthol has a vapor pressure of approximately 7.67×10−3 mm Hg (approximately 1.02 Pa). At atmospheric pressure (760 mmHg or 101325 Pa) menthol has a boiling point of 212° C. This volatility gives rise to menthol's minty odor, but is detrimental to the storage stability of liquid containing menthol. In particular, the shelf life of a liquid is significantly reduced by the presence of menthol because it readily volatilizes under typical storage conditions, e.g. under ambient pressure and temperature as applied by a user to an e-liquid cartridge.

We have found that by incorporating a cooling agent which is less volatile at atmospheric pressure than menthol and optionally a compound of formula (I) or a salt and/or solvate thereof, a vaporizable formulation may be provided which has improved storage stability and hence a longer shelf-life compared to a vaporizable formulation containing menthol. The cooling agents described herein also deliver a cooling sensation when a vapor is generated by a user from the vaporizable formulation and have been shown to introduce different sensory benefits compared to menthol.

For ease of reference, these and further aspects of the present invention are now discussed under appropriate section headings. However, the teachings under each section are not necessarily limited to each particular section.

Cooling Agent

As discussed herein, the vaporizable formulation of the first characteristic of the present invention comprises a cooling agent in an amount of less than about 1 wt % based on the total weight of the vaporizable formulation. By the term “less than” is meant any amount lower than 1 wt % but not including zero. In other words, the vaporizable formulation must include an amount of the cooling agent.

In one aspect the cooling agent is present in an amount of no greater than about 0.9 wt %.

In one aspect the cooling agent is present in an amount of from about 0.001 wt % to about 0.9 wt % based on the total weight of the vaporizable formulation. In one aspect the cooling agent is present in an amount of about 0.002 wt % to about 0.9 wt % based on the total weight of the vaporizable formulation. In one aspect the cooling agent is present in an amount of about 0.003 wt % to about 0.9 wt % based on the total weight of the vaporizable formulation. In one aspect the cooling agent is present in an amount of from about 0.004 wt % to about 0.9 wt % based on the total weight of the vaporizable formulation. In one aspect the cooling agent is present in an amount of from about 0.005 wt % to about 0.9 wt % based on the total weight of the vaporizable formulation. In one aspect the cooling agent is present in an amount of from about 0.006 wt % to about 0.9 wt % based on the total weight of the vaporizable formulation. In one aspect the cooling agent is present in an amount of from about 0.007 wt % to about 0.9 wt % based on the total weight of the vaporizable formulation. In one aspect the cooling agent is present in an amount of from about 0.008 wt % to about 0.9 wt % based on the total weight of the vaporizable formulation. In one aspect the cooling agent is present in an amount of from about 0.009 wt % to about 0.9 wt % based on the total weight of the vaporizable formulation.

In one aspect the cooling agent is present in an amount of from about 0.01 wt % to about 0.9 wt % based on the total weight of the vaporizable formulation. In one aspect the cooling agent is present in an amount of from about 0.02 wt % to about 0.9 wt % based on the total weight of the vaporizable formulation. In one aspect the cooling agent is present in an amount of from about 0.03 wt % to about 0.9 wt % based on the total weight of the vaporizable formulation. In one aspect the cooling agent is present in an amount of from about 0.04 wt % to about 0.9 wt % based on the total weight of the vaporizable formulation. In one aspect the cooling agent is present in an amount of from about 0.05 wt % to about 0.9 wt % based on the total weight of the vaporizable formulation. In one aspect the cooling agent is present in an amount of from 0.06 wt % to about 0.9 wt % based on the total weight of the vaporizable formulation. In one aspect the cooling agent is present in an amount of from about 0.07 wt % to about 0.9 wt % based on the total weight of the vaporizable formulation. In one aspect the cooling agent is present in an amount of from about 0.08 wt % to about 0.9 wt % based on the total weight of the vaporizable formulation. In one aspect the cooling agent is present in an amount of from about 0.09 wt % to about 0.9 wt % based on the total weight of the vaporizable formulation.

In one aspect the cooling agent is present in an amount of from about 0.1 wt % to about 0.9 wt % based on the total weight of the vaporizable formulation. In one aspect the cooling agent is present in an amount of from about 0.2 wt % to about 0.9 wt % based on the total weight of the vaporizable formulation. In one aspect the cooling agent is present in an amount of from about 0.3 wt % to about 0.9 wt % based on the total weight of the vaporizable formulation. In one aspect the cooling agent is present in an amount of from about 0.4 wt % to about 0.9 wt % based on the total weight of the vaporizable formulation. In one aspect the cooling agent is present in an amount of from about 0.5 wt % to about 0.9 wt % based on the total weight of the vaporizable formulation.

In another aspect the cooling agent is present in an amount of no greater than about 0.8 wt %.

In one aspect the cooling agent is present in an amount of from about 0.001 wt % to about 0.8 wt % based on the total weight of the vaporizable formulation. In one aspect the cooling agent is present in an amount of from about 0.002 wt % to about 0.8 wt % based on the total weight of the vaporizable formulation. In one aspect the cooling agent is present in an amount of from about 0.003 wt % to about 0.8 wt % based on the total weight of the vaporizable formulation. In one aspect the cooling agent is present in an amount of from about 0.004 wt % to about 0.8 wt % based on the total weight of the vaporizable formulation. In one aspect the cooling agent is present in an amount of from about 0.005 wt % to about 0.8 wt % based on the total weight of the vaporizable formulation. In one aspect the cooling agent is present in an amount of from about 0.006 wt % to about 0.8 wt % based on the total weight of the vaporizable formulation. In one aspect the cooling agent is present in an amount of from about 0.007 wt % to about 0.8 wt % based on the total weight of the vaporizable formulation. In one aspect the cooling agent is present in an amount of from about 0.008 wt % to about 0.8 wt % based on the total weight of the vaporizable formulation. In one aspect the cooling agent is present in an amount of from about 0.009 wt % to about 0.8 wt % based on the total weight of the vaporizable formulation.

In one aspect the cooling agent is present in an amount of from about 0.01 wt % to about 0.8 wt % based on the total weight of the vaporizable formulation. In one aspect the cooling agent is present in an amount of from about 0.02 wt % to about 0.8 wt % based on the total weight of the vaporizable formulation. In one aspect the cooling agent is present in an amount of from about 0.03 wt % to about 0.8 wt % based on the total weight of the vaporizable formulation. In one aspect the cooling agent is present in an amount of from about 0.04 wt % to about 0.8 wt % based on the total weight of the vaporizable formulation. In one aspect the cooling agent is present in an amount of from about 0.05 wt % to about 0.8 wt % based on the total weight of the vaporizable formulation. In one aspect the cooling agent is present in an amount of from about 0.06 wt % to about 0.8 wt % based on the total weight of the vaporizable formulation. In one aspect the cooling agent is present in an amount of from about 0.07 wt % to about 0.8 wt % based on the total weight of the vaporizable formulation. In one aspect the cooling agent is present in an amount of from about 0.08 wt % to about 0.8 wt % based on the total weight of the vaporizable formulation. In one aspect the cooling agent is present in an amount of from about 0.09 wt % to about 0.8 wt % based on the total weight of the vaporizable formulation.

In one aspect the cooling agent is present in an amount of from about 0.1 wt % to about 0.8 wt % based on the total weight of the vaporizable formulation. In one aspect the cooling agent is present in an amount of from about 0.2 wt % to about 0.8 wt % based on the total weight of the vaporizable formulation. In one aspect the cooling agent is present in an amount of from about 0.3 wt % to about 0.8 wt % based on the total weight of the vaporizable formulation. In one aspect the cooling agent is present in an amount of from about 0.4 wt % to about 0.8 wt % based on the total weight of the vaporizable formulation.

In another aspect the cooling agent is present in an amount of no greater than about 0.75 wt %.

In one aspect the cooling agent is present in an amount of from about 0.001 wt % to about 0.75 wt % based on the total weight of the vaporizable formulation. In one aspect the cooling agent is present in an amount of from about 0.002 wt % to about 0.75 wt % based on the total weight of the vaporizable formulation. In one aspect the cooling agent is present in an amount of from about 0.003 wt % to about 0.75 wt % based on the total weight of the vaporizable formulation. In one aspect the cooling agent is present in an amount of from about 0.004 wt % to about 0.75 wt % based on the total weight of the vaporizable formulation. In one aspect the cooling agent is present in an amount of from about 0.005 wt % to about 0.75 wt % based on the total weight of the vaporizable formulation. In one aspect the cooling agent is present in an amount of from about 0.006 wt % to about 0.75 wt % based on the total weight of the vaporizable formulation. In one aspect the cooling agent is present in an amount of from about 0.007 wt % to about 0.75 wt % based on the total weight of the vaporizable formulation. In one aspect the cooling agent is present in an amount of from about 0.008 wt % to about 0.75 wt % based on the total weight of the vaporizable formulation. In one aspect the cooling agent is present in an amount of from about 0.009 wt % to about 0.75 wt % based on the total weight of the vaporizable formulation.

In one aspect the cooling agent is present in an amount of from about 0.01 wt % to about 0.75 wt % based on the total weight of the vaporizable formulation. In one aspect the cooling agent is present in an amount of from about 0.02 wt % to about 0.75 wt % based on the total weight of the vaporizable formulation. In one aspect the cooling agent is present in an amount of from about 0.03 wt % to about 0.75 wt % based on the total weight of the vaporizable formulation. In one aspect the cooling agent is present in an amount of from about 0.04 wt % to about 0.75 wt % based on the total weight of the vaporizable formulation. In one aspect the cooling agent is present in an amount of from about 0.05 wt % to about 0.75 wt % based on the total weight of the vaporizable formulation. In one aspect the cooling agent is present in an amount of from about 0.06 wt % to about 0.75 wt % based on the total weight of the vaporizable formulation. In one aspect the cooling agent is present in an amount of from about 0.07 wt % to about 0.75 wt % based on the total weight of the vaporizable formulation. In one aspect the cooling agent is present in an amount of from about 0.08 wt % to about 0.75 wt % based on the total weight of the vaporizable formulation. In one aspect the cooling agent is present in an amount of from about 0.09 wt % to about 0.75 wt % based on the total weight of the vaporizable formulation.

In one aspect the cooling agent is present in an amount of from about 0.1 wt % to about 0.75 wt % based on the total weight of the vaporizable formulation. In one aspect the cooling agent is present in an amount of from about 0.2 wt % to about 0.75 wt % based on the total weight of the vaporizable formulation. In one aspect the cooling agent is present in an amount of from about 0.3 wt % to about 0.75 wt % based on the total weight of the vaporizable formulation. In one aspect the cooling agent is present in an amount of from about 0.4 wt % to about 0.75 wt % based on the total weight of the vaporizable formulation.

In one aspect the cooling agent is present in an amount of no greater than about 0.5 wt %.

In one aspect the cooling agent is present in an amount of from about 0.001 wt % to about 0.5 wt % based on the total weight of the vaporizable formulation. In one aspect the cooling agent is present in an amount of from about 0.002 wt % to about 0.5 wt % based on the total weight of the vaporizable formulation. In one aspect the cooling agent is present in an amount of from about 0.003 wt % to about 0.5 wt % based on the total weight of the vaporizable formulation. In one aspect the cooling agent is present in an amount of from about 0.004 wt % to about 0.5 wt % based on the total weight of the vaporizable formulation. In one aspect the cooling agent is present in an amount of from about 0.005 wt % to about 0.5 wt % based on the total weight of the vaporizable formulation. In one aspect the cooling agent is present in an amount of from about 0.006 wt % to about 0.5 wt % based on the total weight of the vaporizable formulation. In one aspect the cooling agent is present in an amount of from about 0.007 wt % to about 0.5 wt % based on the total weight of the vaporizable formulation. In one aspect the cooling agent is present in an amount of from about 0.008 wt % to about 0.5 wt % based on the total weight of the vaporizable formulation. In one aspect the cooling agent is present in an amount of from about 0.009 wt % to about 0.5 wt % based on the total weight of the vaporizable formulation. In one aspect the cooling agent is present in an amount of from about 0.01 wt % to about 0.5 wt % based on the total weight of the vaporizable formulation. In one aspect the cooling agent is present in an amount of from about 0.02 wt % to about 0.5 wt % based on the total weight of the vaporizable formulation. In one aspect the cooling agent is present in an amount of from about 0.03 wt % to about 0.5 wt % based on the total weight of the vaporizable formulation. In one aspect the cooling agent is present in an amount of from about 0.04 wt % to about 0.5 wt % based on the total weight of the vaporizable formulation. In one aspect the cooling agent is present in an amount of from about 0.05 wt % to about 0.5 wt % based on the total weight of the vaporizable formulation. In one aspect the cooling agent is present in an amount of from about 0.06 wt % to about 0.5 wt % based on the total weight of the vaporizable formulation. In one aspect the cooling agent is present in an amount of from about 0.07 wt % to about 0.5 wt % based on the total weight of the vaporizable formulation. In one aspect the cooling agent is present in an amount of from about 0.08 wt % to about 0.5 wt % based on the total weight of the vaporizable formulation. In one aspect the cooling agent is present in an amount of from about 0.09 wt % to about 0.5 wt % based on the total weight of the vaporizable formulation.

In one aspect the cooling agent is present in an amount of from about 0.1 wt % to about 0.5 wt % based on the total weight of the vaporizable formulation. In one aspect the cooling agent is present in an amount of from about 0.2 wt % to about 0.5 wt % based on the total weight of the vaporizable formulation. In one aspect the cooling agent is present in an amount of from about 0.3 wt % to about 0.5 wt % based on the total weight of the vaporizable formulation. In one aspect the cooling agent is present in an amount of from about 0.4 wt % to about 0.5 wt % based on the total weight of the vaporizable formulation.

In another aspect the cooling agent is present in an amount of no greater than about 0.3 wt %.

In one aspect the cooling agent is present in an amount of from about 0.001 wt % to about 0.3 wt % based on the total weight of the vaporizable formulation. In one aspect the cooling agent is present in an amount of from about 0.002 wt % to about 0.3 wt % based on the total weight of the vaporizable formulation. In one aspect the cooling agent is present in an amount of from about 0.003 wt % to about 0.3 wt % based on the total weight of the vaporizable formulation. In one aspect the cooling agent is present in an amount of from about 0.004 wt % to about 0.3 wt % based on the total weight of the vaporizable formulation. In one aspect the cooling agent is present in an amount of from about 0.005 wt % to about 0.3 wt % based on the total weight of the vaporizable formulation. In one aspect the cooling agent is present in an amount of from about 0.006 wt % to about 0.3 wt % based on the total weight of the vaporizable formulation. In one aspect the cooling agent is present in an amount of from about 0.007 wt % to about 0.3 wt % based on the total weight of the vaporizable formulation. In one aspect the cooling agent is present in an amount of from about 0.008 wt % to about 0.3 wt % based on the total weight of the vaporizable formulation. In one aspect the cooling agent is present in an amount of from about 0.009 wt % to about 0.3 wt % based on the total weight of the vaporizable formulation.

In one aspect the cooling agent is present in an amount of from about 0.01 wt % to about 0.3 wt % based on the total weight of the vaporizable e formulation. In one aspect the cooling agent is present in an amount of from about 0.02 wt % to about 0.3 wt % based on the total weight of the vaporizable formulation. In one aspect the cooling agent is present in an amount of from about 0.03 wt % to about 0.3 wt % based on the total weight of the vaporizable formulation. In one aspect the cooling agent is present in an amount of from about 0.04 wt % to about 0.3 wt % based on the total weight of the vaporizable formulation. In one aspect the cooling agent is present in an amount of from about 0.05 wt % to about 0.3 wt % based on the total weight of the vaporizable formulation. In one aspect the cooling agent is present in an amount of from about 0.06 wt % to about 0.3 wt % based on the total weight of the vaporizable formulation. In one aspect the cooling agent is present in an amount of from about 0.07 wt % to about 0.3 wt % based on the total weight of the vaporizable formulation. In one aspect the cooling agent is present in an amount of from about 0.08 wt % to about 0.3 wt % based on the total weight of the vaporizable formulation. In one aspect the cooling agent is present in an amount of from about 0.09 wt % to about 0.3 wt % based on the total weight of the vaporizable formulation.

In one aspect the cooling agent is present in an amount of from about 0.1 wt % to about 0.3 wt % based on the total weight of the vaporizable formulation. In one aspect the cooling agent is present in an amount of from about 0.2 wt % to about 0.3 wt % based on the total weight of the vaporizable formulation.

It will be understood by the skilled person that the vaporizable formulation described herein can include more than one cooling agent. When the formulation includes more than one cooling agent, each cooling agent can be included at the above defined amounts, such that each cooling agent is included in an amount of less than about 1 wt %. For example, a first cooling agent may be included in an amount of about 0.001 wt % to 0.9 wt % based on the weight of the vaporizable formulation and a second cooling agent may be included in an amount of about 0.001 wt % to 0.9 wt % based on the weight of the vaporizable formulation. Alternatively the second cooling agent may be included in an amount of about 0.05 wt % to 0.9 wt % based on the weight of the vaporizable formulation. It being understood that these combination of ranges are purely for example purposes, the vaporizable formulation is not limited in this respect.

In the second characteristic of the present invention, the cooling agent is defined as a compound of formula (I) or a salt and/or solvate thereof, and the vaporizable formulation includes said cooling agent in an amount of less than about 12 wt % based on the total weight of the vaporizable formulation. The term “less than” has the same meaning as in the first characteristic. The following disclosure relates to the second characteristic.

In one aspect, said cooling agent of the second characteristic is present in an amount of no greater than about 11 wt %.

In one aspect said cooling agent is present in an amount of from about 0.001 wt % to about 11 wt % based on the total weight of the vaporizable formulation. In one aspect said cooling agent is present in an amount of about 0.005 wt % to about 11 wt % based on the total weight of the vaporizable formulation. In one aspect said cooling agent is present in an amount of about 0.01 wt % to about 11 wt % based on the total weight of the vaporizable formulation. In one aspect said cooling agent is present in an amount of from about 0.05 wt % to about 11 wt % based on the total weight of the vaporizable formulation. In one aspect said cooling agent is present in an amount of from about 0.1 wt % to about 11 wt % based on the total weight of the vaporizable formulation.

In another aspect said cooling agent is present in an amount of no greater than about 10 wt %.

In one aspect said cooling agent is present in an amount of from about 0.001 wt % to about 10 wt % based on the total weight of the vaporizable formulation. In one aspect said cooling agent is present in an amount of about 0.005 wt % to about 10 wt % based on the total weight of the vaporizable formulation. In one aspect said cooling agent is present in an amount of about 0.01 wt % to about 10 wt % based on the total weight of the vaporizable formulation. In one aspect said cooling agent is present in an amount of from about 0.05 wt % to about 10 wt % based on the total weight of the vaporizable formulation. In one aspect said cooling agent is present in an amount of from about 0.1 wt % to about 10 wt % based on the total weight of the vaporizable formulation.

In another aspect said cooling agent is present in an amount of no greater than about 8 wt %.

In one aspect said cooling agent is present in an amount of from about 0.001 wt % to about 8 wt % based on the total weight of the vaporizable formulation. In one aspect said cooling agent is present in an amount of about 0.005 wt % to about 8 wt % based on the total weight of the vaporizable formulation. In one aspect said cooling agent is present in an amount of about 0.01 wt % to about 8 wt % based on the total weight of the vaporizable formulation. In one aspect said cooling agent is present in an amount of from about 0.05 wt % to about 8 wt % based on the total weight of the vaporizable formulation. In one aspect said cooling agent is present in an amount of from about 0.1 wt % to about 8 wt % based on the total weight of the vaporizable formulation.

In another aspect said cooling agent is present in an amount of no greater than about 5 wt %.

In one aspect said cooling agent is present in an amount of from about 0.001 wt % to about 5 wt % based on the total weight of the vaporizable formulation. In one aspect said cooling agent is present in an amount of about 0.005 wt % to about 5 wt % based on the total weight of the vaporizable formulation. In one aspect said cooling agent is present in an amount of about 0.01 wt % to about 5 wt % based on the total weight of the vaporizable formulation. In one aspect said cooling agent is present in an amount of from about 0.05 wt % to about 5 wt % based on the total weight of the vaporizable formulation. In one aspect said cooling agent is present in an amount of from about 0.1 wt % to about 5 wt % based on the total weight of the vaporizable formulation.

In another aspect said cooling agent is present in an amount of no greater than about 3 wt %, e.g. about 2.5 wt %.

In one aspect said cooling agent is present in an amount of from about 0.001 wt % to about 3 wt % based on the total weight of the vaporizable formulation. In one aspect said cooling agent is present in an amount of about 0.005 wt % to about 3 wt % based on the total weight of the vaporizable formulation. In one aspect said cooling agent is present in an amount of about 0.01 wt % to about 3 wt % based on the total weight of the vaporizable formulation. In one aspect said cooling agent is present in an amount of from about 0.05 wt % to about 3 wt % based on the total weight of the vaporizable formulation. In one aspect said cooling agent is present in an amount of from about 0.1 wt % to about 3 wt % based on the total weight of the vaporizable formulation.

As for the first characteristic, it will be understood by the skilled person that the vaporizable formulation described herein for the second characteristic can include more than one cooling agent. When the formulation includes more than one cooling agent, each cooling agent can be included at the above defined amounts, such that each cooling gent is included in an amount of less than about 12 wt %. Alternatively, one cooling agent could be included in an amount of less than about 12 wt % according to the second characteristic of the invention, and the second cooling agent could be included in an amount of less than about 1 wt %, according to the first characteristic of the invention. It being understood that these combination of ranges are purely for example purposes, the vaporizable formulation not being limited in this respect.

In both the first and second characteristics, the cooling agent is a compound which volatilizes at a higher temperature than menthol at atmospheric pressure. By the term “cooling agent” is meant a compound which delivers a cooling or fresh sensation to a user when inhaled as a vapour.

By the term “volatilizes” is meant the physical change of the compound from a liquid state into a gaseous state. The term “volatilizes” can be used interchangeably with “vaporizes” Vaporization or volatilization of a compound is a phase transition from the liquid phase to vapor and there are two types: evaporation and boiling. Evaporation is a surface phenomenon, whereas boiling is a bulk phenomenon.

Boiling is the formation of vapor as bubbles of vapor below the surface of the liquid, and occurs when the equilibrium vapor pressure of the compound is greater than or equal to the environmental pressure. The temperature at which boiling occurs is the boiling temperature or boiling point. Evaporation occurs at temperatures below the boiling temperature at a given pressure because it occurs when the partial pressure of vapor of a compound is less than the equilibrium vapor pressure.

The term “volatilizes” in the context of the present description refers to evaporation or boiling. Thus the expression “volatilizes” at a higher temperature than menthol at atmospheric pressure” means that at atmospheric pressure the cooling agent transitions to vapor, whether due to evaporation or boiling, at a higher temperature than menthol.

By the term “atmospheric pressure” is meant 101325 Pa, equivalent to 760 mmHg.

In one aspect the cooling agent has a boiling point of above 212° C. at atmospheric pressure. In one aspect the cooling agent has a boiling point of above 220° C. at atmospheric pressure. In one aspect the cooling agent has a boiling point of above 230° C. at atmospheric pressure. In one aspect the cooling agent has a boiling point of above 250° C. at atmospheric pressure. In one aspect the cooling agent has a boiling point of above 300° C. at atmospheric pressure. In one aspect the cooling agent has a boiling point of above 350° C. at atmospheric pressure. In one aspect the cooling agent has a boiling point of above 375° C. at atmospheric pressure. In one aspect the cooling agent has a boiling point of above 400° C. at atmospheric pressure. In one aspect the cooling agent has a boiling point of above 450° C. at atmospheric pressure. In one aspect the cooling agent has a boiling point of above 460° C. at atmospheric pressure.

In one aspect the cooling agent volatilizes at a temperature of above 212° C. at atmospheric pressure. In one aspect the cooling agent volatilizes at a temperature of above 220° C. at atmospheric pressure. In one aspect the cooling agent volatilizes at a temperature of above 230° C. at atmospheric pressure. In one aspect the cooling agent volatilizes at a temperature of above 250° C. at atmospheric pressure. In one aspect the cooling agent volatilizes at a temperature of above 300° C. at atmospheric pressure. In one aspect the cooling agent volatilizes at a temperature of above 350° C. at atmospheric pressure. In one aspect the cooling agent volatilizes at a temperature of above 375° C. at atmospheric pressure. In one aspect the cooling agent volatilizes at a temperature of above 400° C. at atmospheric pressure. In one aspect the cooling agent volatilizes at a temperature of above 450° C. at atmospheric pressure. In one aspect the cooling agent volatilizes at a temperature of above 460° C. at atmospheric pressure.

In one aspect the cooling agent has a boiling point in the range of about 230° C. to about 500° C. at atmospheric pressure. In one aspect the cooling agent has a boiling point in the range of about 250° C. to about 500° C. at atmospheric pressure. In one aspect the cooling agent has a boiling point in the range of about 300° C. to about 500° C. at atmospheric pressure. In one aspect the cooling agent has a boiling point in the range of about 340° C. to about 500° C. at atmospheric pressure.

In one aspect the cooling agent has a boiling point in the range of about 230° C. to about 465° C. at atmospheric pressure. In one aspect the cooling agent has a boiling point in the range of about 250° C. to about 465° C. at atmospheric pressure. In one aspect the cooling agent has a boiling point in the range of about 300° C. to about 465° C. at atmospheric pressure. In one aspect the cooling agent has a boiling point in the range of about 340° C. to about 465° C. at atmospheric pressure.

In one aspect the cooling agent volatilizes at a temperature in the range of about 230° C. to about 500° C. at atmospheric pressure. In one aspect the cooling agent volatilizes at a temperature in the range of about 250° C. to about 500° C. at atmospheric pressure. In one aspect the cooling agent volatilizes at a temperature in the range of about 300° C. to about 500° C. at atmospheric pressure. In one aspect the cooling agent volatilizes at a temperature in the range of about 340° C. to about 500° C. at atmospheric pressure.

In one aspect the cooling agent volatilizes at a temperature in the range of about 230° C. to about 465° C. at atmospheric pressure. In one aspect the cooling agent volatilizes at a temperature in the range of about 250° C. to about 465° C. at atmospheric pressure. In one aspect the cooling agent volatilizes at a temperature in the range of about 300° C. to about 465° C. at atmospheric pressure. In one aspect the cooling agent volatilizes at a temperature in the range of about 340° C. to about 465° C. at atmospheric pressure.

The temperature at which a compound turns from liquid into vapor can be readily determined by the person skilled in the art using techniques known in the art. A well-known method is distillation such as that described by the JECFA (Joint Expert Committee on Food Additives) on http://www.fao.org/docrep/009/a0691e/a0691e00.htm. This method relies on the use of a distillation thermometer to determine an initial boiling point. If the distillation is not carried at atmospheric pressure then the observed temperature should be corrected, allowing 0.10 for each 2.7 mm of variation.

In one aspect the cooling agent has a vapor pressure lower than menthol at room temperature. In one aspect the cooling agent has a vapor pressure lower than menthol at 25° C., for example a vapor pressure below 7.67×10⁻³ mm Hg (1.02 Pa or 102 mPa).

Vapor pressure is also known as equilibrium vapor pressure and is defined as the pressure exerted by a vapor in thermodynamic equilibrium with its condensed phases (solid or liquid) at a given temperature in a closed system. The vapor pressure is an indication of a liquid's evaporation rate because it relates to the tendency of particles to escape from the liquid (or solid). A substance with a high vapor pressure at normal temperatures, such as menthol, is referred to as volatile.

As is known in the art, vapor pressure is measured by an isoteniscope. This device consists of a submerged manometer and container holding the substance whose vapor pressure is being measured. The liquid in which the manometer is submerged is heated to the required temperature, here 25° C., and the open end of the manometer is connected to a pressure measuring device. A vacuum pump is used to adjust the pressure of the system and purify the sample.

In one aspect the cooling agent has a vapor pressure at 25° C. below 50 mPa. In one aspect the cooling agent has a vapor pressure at 25° C. below 25 mPa. In one aspect the cooling agent has a vapor pressure at 25° C. below 15 mPa. In one aspect the cooling agent has a vapor pressure at 25° C. below 10 mPa. In one aspect the cooling agent has a vapor pressure at 25° C. below 5 mPa. In one aspect the cooling agent has a vapor pressure at 25° C. below 1 mPa. In one aspect the cooling agent has a vapor pressure at 25° C. below 0.5 mPa. In one aspect the cooling agent has a vapor pressure at 25° C. below 0.1 mPa. In one aspect the cooling agent has a vapor pressure at 25° C. below 0.05 mPa. In one aspect the cooling agent has a vapor pressure at 25° C. below 0.01 mPa. In one aspect the cooling agent has a vapor pressure at 25° C. below 0.005 mPa.

In one aspect the cooling agent has a vapor pressure at 25° C. in the range of 0.001 mPa to 15 mPa. In one aspect the cooling agent has a vapor pressure at 25° C. in the range of 0.001 mPa to 12 mPa. In one aspect the cooling agent has a vapor pressure at 25° C. in the range of 0.001 mPa to 10 mPa. In one aspect the cooling agent has a vapor pressure at 25° C. in the range of 0.001 mPa to 8 mPa. In one aspect the cooling agent has a vapor pressure at 25° C. in the range of 0.001 mPa to 5 mPa. In one aspect the cooling agent has a vapor pressure at 25° C. in the range of 0.001 mPa to 3 mPa. In one aspect the cooling agent has a vapor pressure at 25° C. in the range of 0.001 mPa to 1 mPa. In one aspect the cooling agent has a vapor pressure at 25° C. in the range of 0.001 mPa to 0.5 mPa. In one aspect the cooling agent has a vapor pressure at 25° C. in the range of 0.001 mPa to 0.1 mPa. In one aspect the cooling agent has a vapor pressure at 25° C. in the range of 0.001 mPa to 0.003 mPa.

In one aspect the cooling agent is a compound of formula (I) or a salt and/or solvate thereof:

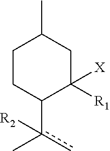

wherein X is hydrogen or OR′, wherein R′ is an alkyl group or an alkenyl group which may be taken together with R₁ to form a three to five-membered heterocyclyl group, wherein the heterocyclyl group is optionally substituted by one or more substituents selected from OH, O-alkyl, alkyl-OH, alkyl-O-alkyl, NH₂, NH-alkyl, N-(alkyl)₂, NO₂ and CN; and wherein R₁ and R₂ are each independently selected from hydrogen, OH, ORₐ, C(O)NR_{b}R_{c} and C(O)OR_{b}R_{c}; with the proviso that when R₁ is OH the compound of formula (I) is not menthol; and when the double bond is present, R₂ is absent;

wherein Rₐ is an alkyl group, an alkenyl group, a C(O)R_{f} group, or a C(O)-alkyl-C(O)R_{f} group wherein the alkyl groups and alkenyl groups are optionally substituted by one or more substituents selected from OH, O-alkyl, NH₂, NH-alkyl, N-(alkyl)₂, NO₂ and CN; and wherein R_{f} is an alkyl group, an alkenyl group, OH, O-alkyl, NH₂, NH-alkyl or N-(alkyl)₂, wherein the alkyl groups and alkenyl groups are optionally substituted by one or more substituents selected from OH, O-alkyl, NH₂, NH-alkyl, N-(alkyl)₂, NO₂ and CN;

wherein R_{b} and R_{c} are each independently hydrogen, an alkyl group, an alkenyl group, an aryl group, an aralkyl group, a heteroaryl group, or a heteroaralkyl group, wherein the alkyl groups, alkenyl groups, aryl groups and heteroaryl groups are optionally substituted by one or more substituents selected from OH, O-alkyl, NH₂, NH-alkyl, N-(alkyl)₂, NO₂, CN and C(O)R_{f}.

In one aspect X is hydrogen.

In one aspect X is OR′, wherein R′ is an alkyl group or an alkenyl group which is taken together with R₁ to form a three to five-membered heterocyclyl group, wherein the heterocyclyl group is optionally substituted by OH, O-alkyl or alkyl-OH. In one aspect X is OR′, wherein R′ is an alkyl group which is taken together with R₁ to form a four or five-membered heterocyclyl group, wherein the heterocyclyl group is optionally substituted by alkyl-OH. In one aspect X is OR′, wherein R′ is an alkyl group which is taken together with R₁ to form a four or five-membered heterocyclyl group, wherein the heterocyclyl group is optionally substituted by alkyl-OH, and wherein R₁ is ORₐ wherein Rₐ is an alkyl group and wherein R₂ is absent or hydrogen.

In one aspect R₁ is selected from OH, ORₐ and C(O)NR_{b}R_{c} and R₂ is either absent or selected from OH and ORₐ. In one aspect R₁ is OH with the proviso that the compound of formula (I) is not menthol. In one aspect R₁ is OH and R₂ is selected from OH and ORₐ.

In one aspect X is hydrogen and R₁ is selected from OH, ORₐ and C(O)NR_{b}R_{c}, with the proviso that, when R₁ is OH, the compound of formula (I) is not menthol. R₂ is either absent or selected from OH and ORₐ. In one aspect X is hydrogen, R₁ is selected from ORₐ and C(O)NR_{b}R_{c} and R₂ is either absent or selected from OH and ORₐ.

In one aspect R₁ is ORₐ and Rₐ is an alkyl group substituted by one or more OH substituents. R₂ may be hydrogen.

In one aspect R₁ is ORₐ and Rₐ is a C(O)R_{f} group, or a C(O)-alkyl-C(O)R_{f} group, wherein R_{f} is an alkyl group optionally substituted by one or more OH substituents or R_{f} is OH. R₂ may be hydrogen.

In one aspect R₁ is C(O)NR_{b}R_{c}, wherein R_{b} and R_{c} are each independently hydrogen, an alkyl group, an aryl group, an aralkyl group, a heteroaryl group, or a heteroaralkyl group. In one aspect R₁ is C(O)NR_{b}R_{c} and at least one of R_{b} and R_{c} is hydrogen. R₂ may be hydrogen.

In one aspect R₁ is C(O)NR_{b}R_{c}, wherein R_{b} is hydrogen and Re is selected from the group consisting of an alkyl group, an aryl group, an aralkyl group and a heteroaralkyl group. R₂ may be hydrogen.

As used herein, the term “alkyl” includes both saturated straight chain and branched alkyl groups which may be substituted (mono- or poly-) or unsubstituted. In one aspect the alkyl group is a C₁₋₁₀ alkyl group. In one aspect the alkyl group is a C₁₋₈ alkyl group. In one aspect the alkyl group is a C₁₋₆ alkyl group. In one aspect the alkyl group is a C₁₋₃ alkyl group. In one aspect the alkyl groups include, for example, methyl, ethyl, propyl, isopropyl, butyl, isobutyl, tert-butyl, pentyl and hexyl. In one aspect the alkyl groups include methyl, ethyl, propyl or isopropyl.

As used herein, the term “alkenyl” includes both unsaturated straight chain and branched alkenyl groups which may be substituted (mono- or poly-) or unsubstituted. In one aspect the alkenyl group is a C₂₋₁₀ alkenyl group. In one aspect the alkenyl group is a C₂₋₈ alkenyl group. In one aspect the alkenyl group is a C₂₋₆ alkenyl group. In one aspect the alkenyl group is a C₂₋₃ alkenyl group.

As used herein, the term “aryl” refers to a C₆₋₁₂ aromatic group which may be substituted (mono- or poly-) or unsubstituted. Typical examples include phenyl and naphthyl etc. In one aspect the aryl group is phenyl.

The term “aralkyl” is used as a conjunction of the terms alkyl and aryl as given above. For example an aryl group may be bonded to the compound of formula (I) through a diradical alkylene bridge, (—CH₂—)ₙ, where n is 1-10 and where “aryl” is as defined above. Alternatively an alkyl group may be bonded to the compound of formula (I) through a diradical aryl bridge, e.g. phenyl, where “alkyl is as defined above. In one aspect the term “aralkyl” refers to a phenyl-alkyl group where the phenyl is bonded to the compound of formula (I).

As used herein the term “heteroaryl” refers to a monovalent aromatic group of from 1 to 12 carbon atoms having one or more oxygen, nitrogen, and sulfur heteroatoms within the ring. In one aspect there are 1 to 4 oxygen, nitrogen and/or sulfur heteroatoms within the ring. In one aspect there are 1 to 3 oxygen, nitrogen and/or sulfur heteroatoms within the ring. In one aspect there are 2 oxygen and/or nitrogen heteroatoms within the ring. In one aspect there is 1 oxygen or nitrogen heteroatom within the ring. The nitrogen and sulfur heteroatoms may optionally be oxidized. Such heteroaryl groups can have a single ring (e.g., pyridyl or furyl) or multiple condensed rings provided that the point of attachment is through a heteroaryl ring atom.

In one aspect the heteroaryl is selected from the group consisting of pyridyl, pyridazinyl, pyrimidinyl, pyrazinyl, triazinyl, pyrrolyl, indolyl, quinolinyl, isoquinolinyl, quinazolinyl, quinoxalinnyl, furanyl, thiophenyl, furyl, pyrrolyl, imidazolyl, oxazolyl, isoxazolyl, isothiazolyl, pyrazolyl benzofuranyl, and benzothiophenyl. Heteroaryl rings may be unsubstituted or substituted. In one aspect the heteroaryl is selected from the group consisting of pyridyl, pyridazinyl, pyrimidinyl, pyrazinyl, triazinyl and pyrrolyl. In one aspect the heteroaryl is pyridyl.

As used herein the term “heterocyclyl” refers to fully saturated or unsaturated, monocyclic groups, which have one or more oxygen, sulfur or nitrogen heteroatoms in the ring. In one aspect the heterocyclyl has 1 to 3 heteroatoms in the ring. In one aspect the heterocyclyl has 1 to 3 oxygen and/or nitrogen heteroatoms in the ring. In one aspect the heterocyclyl has 1 to 3 oxygen heteroatoms in the ring. The nitrogen and sulfur heteroatoms may optionally be oxidized and the nitrogen heteroatoms may optionally be quaternized. The heterocyclic group may be unsubstituted or substituted.

Exemplary monocyclic heterocyclic groups include, but are not limited to, pyrrolidinyl, pyrrolyl, pyrazolyl, oxiranyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, triazolyl, and triazinyl.

In one aspect the heterocycyl is selected from the group consisting of oxiranyl, oxetanyl, tetrahydrofuryl, tetrahydropyranyl, and 1,3-dioxolane. In one aspect the heterocycyl is 1,3-dioxolane.

All aspects include, where appropriate, all enantiomers and tautomers of the compounds of formula (I). The man skilled in the art will recognise compounds that possess optical properties (one or more chiral carbon atoms) or tautomeric characteristics. The corresponding enantiomers and/or tautomers may be isolated/prepared by methods known in the art. Some of the compounds of formula (I) may exist as stereoisomers and/or geometric isomers—e.g. they may possess one or more asymmetric and/or geometric centers and so may exist in two or more stereoisomeric and/or geometric forms. All aspects include, where appropriate, the use of all the individual stereoisomers and geometric isomers of those compounds, and mixtures thereof. The terms used in the claims encompass these forms.

Suitable salts of the compounds of formula (I) include suitable acid addition or base salts thereof. Such salts and solvates thereof will be known in the art. Suitable acid addition salts include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, a-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfite, phosphate, monohydrogenphosphate, dihydrogenphosphate, metaphosphate, pyrophosphate or nitrate salts.

In one aspect, the cooling agent is selected from the group consisting of
N-ethyl-5-methyl-2-(propan-2-yl) cyclohexanecarboxamide,
ethyl-2-(5-methyl-2-propan-2-yl cyclohexanecarbonyl amino) acetate,
N-(4-methoxyphenyl)-p-menthanecarboxamide,
N-2,3-trimethyl-2-propan-2-yl butanamide,
N-(2-pyridin-2-yl)ethyl)menthyl carboxamide,
menthone-1,2-glycerol ketal,
menthyl lactate,
isopulegol,
3-menthoxypropan-1,2-diol, and
menthyl succinate.

In one aspect the cooling agent is selected from the group consisting of
N-ethyl-5-methyl-2-(propan-2-yl) cyclohexanecarboxamide,
ethyl-2-(5-methyl-2-propan-2-yl cyclohexanecarbonyl amino) acetate,
N-(4-methoxyphenyl)-p-menthanecarboxamide,
N-2,3-trimethyl-2-propan-2-yl butanamide,
N-(2-pyridin-2-yl)ethyl)menthyl carboxamide,
menthone-1,2-glycerol ketal,
menthyl lactate,
3-menthoxypropan-1,2-diol, and
menthyl succinate.

In the second characteristic, the cooling agent may be selected from the group consisting of
N-ethyl-5-methyl-2-(propan-2-yl) cyclohexanecarboxamide,
ethyl-2-(5-methyl-2-propan-2-yl cyclohexanecarbonyl amino) acetate,
N-(4-methoxyphenyl)-p-menthanecarboxamide,
N-(2-pyridin-2-yl)ethyl)menthyl carboxamide,
menthone-1,2-glycerol ketal,
menthyl lactate,
isopulegol,
3-menthoxypropan-1,2-diol, and
menthyl succinate,
or
from the group consisting of
N-ethyl-5-methyl-2-(propan-2-yl) cyclohexanecarboxamide,
ethyl-2-(5-methyl-2-propan-2-yl cyclohexanecarbonyl amino) acetate,
N-(4-methoxyphenyl)-p-menthanecarboxamide,
N-(2-pyridin-2-yl)ethyl)menthyl carboxamide,
menthone-1,2-glycerol ketal,
menthyl lactate,
3-menthoxypropan-1,2-diol, and
menthyl succinate.

In one aspect of the first characteristic, the cooling agent is selected from the group consisting of

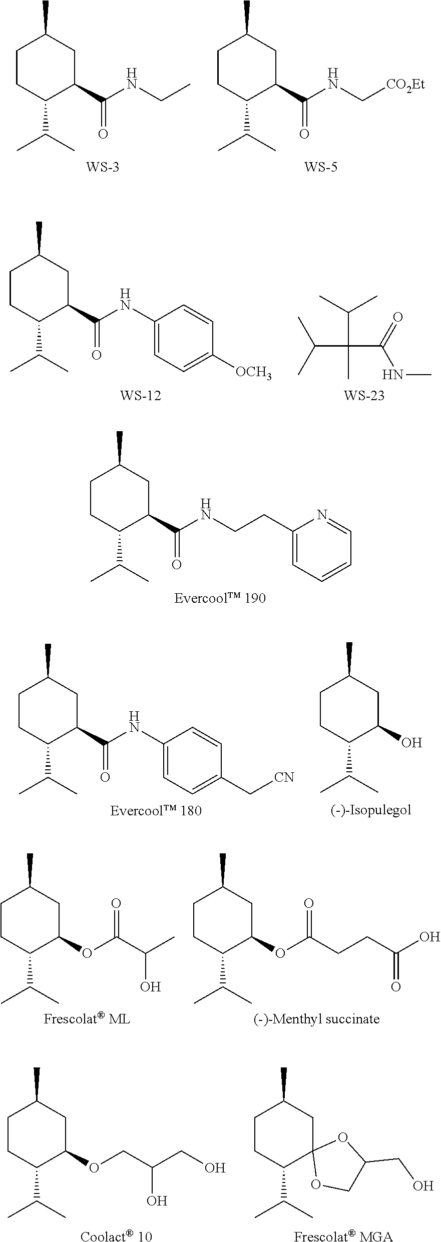

In one aspect the cooling agent is not WS-23, i.e. N,2,3-trimethyl-2-propan-2-ylbutanamide.

In one aspect of the first characteristic, the cooling agent is WS-23, i.e. N,2-3-trimethyl-2-propan-2-ylbutanamide. WS-23 is not encompassed by the second characteristic of the invention.

In one aspect the cooling agent is selected from the group consisting of (1S,2R,5S)-N-ethyl-5-methyl-2-(propan-2-yl)cyclohexanecarboxamide, ethyl-2-[[(1R,2S,5R)-5-methyl-2-propan-2-ylcyclohexanecarbonyl]amino] acetate, (1R,2S,5R)-N-(4-methoxyphenyl-p-menthanecarboxamide, (1R,2S,5R)-N-(2-(pyridin-2-yl)ethyl)menthylcarboxamide, (−)-menthone 1,2-glycerol ketal, (−)-menthyl lactate, (−)-isopulegol, 3-((−)-menthoxy)propane-1,2-diol, and (−)-menthyl succinate.

In one aspect the cooling agent is selected from the group consisting of (1S,2R,5S)-N-ethyl-5-methyl-2-(propan-2-yl)cyclohexanecarboxamide, ethyl-2-[[(1R,2S,5R)-5-methyl-2-propan-2-ylcyclohexanecarbonyl]amino] acetate, (1R,2S,5R)-N-(4-methoxyphenyl-p-menthanecarboxamide, (1R,2S,5R)-N-(2-(pyridin-2-yl)ethyl)menthylcarboxamide, (−)-menthone 1,2-glycerol ketal, (−)-menthyl lactate, 3-((−)-menthoxy)propane-1,2-diol, and (−)-menthyl succinate.

In one aspect the cooling agent is selected from the group consisting of (1S,2R,5S)-N-ethyl-5-methyl-2-(propan-2-yl)cyclohexanecarboxamide, ethyl-2-[[(1R,2S,5R)-5-methyl-2-propan-2-ylcyclohexanecarbonyl]amino] acetate, (1R,2S,5R)-N-(4-methoxyphenyl-p-menthanecarboxamide, (1R,2S,5R)-N-(2-(pyridin-2-yl)ethyl)menthylcarboxamide, (−)-menthone 1,2-glycerol ketal, (−)-menthyl lactate, (−)-isopulegol, and 3-((−)-menthoxy)propane-1,2-diol.

In one aspect the cooling agent is selected from the group consisting of (1S,2R,5S)-N-ethyl-5-methyl-2-(propan-2-yl)cyclohexanecarboxamide, ethyl-2-[[(1R,2S,5R)-5-methyl-2-propan-2-ylcyclohexanecarbonyl]amino] acetate, ((1R,2S,5R)-N-(2-(pyridin-2-yl)ethyl)menthylcarboxamide, (−)-menthone 1,2-glycerol ketal, (−)-menthyl lactate, (−)-isopulegol, and 3-((−)-menthoxy)propane-1,2-diol.

In one aspect the cooling agent is selected from the group consisting of (1S,2R,5S)-N-ethyl-5-methyl-2-(propan-2-yl)cyclohexanecarboxamide, ethyl-2-[[(1R,2S,5R)-5-methyl-2-propan-2-ylcyclohexanecarbonyl]amino] acetate, ((1R,2S,5R)-N-(2-(pyridin-2-yl)ethyl)menthylcarboxamide, (−)-menthone 1,2-glycerol ketal, (−)-menthyl lactate, and 3-((−)-menthoxy)propane-1,2-diol.

In one aspect the cooling agent is (1R,2S,5R)-N-(2-(pyridin-2-yl)ethyl)menthylcarboxamide.

In another aspect the cooling agent is (1S,2R,5S)-N-ethyl-5-methyl-2-(propan-2-yl)cyclohexanecarboxamide.

As noted above, all aspects include, where appropriate, all enantiomers and tautomers of the compounds. The man skilled in the art will recognize compounds that possess optical properties (one or more chiral carbon atoms) or tautomeric characteristics. The corresponding enantiomers and/or tautomers may be isolated/prepared by methods known in the art.

Vaporizable Formulation

The vaporizable formulations of the present invention may contain one or more further components. These components may be selected depending on the nature of the formulation.

In one aspect the formulation further comprises a “flavor” or “flavorant”. The terms “flavor” and “flavorant” refer to materials which, where local regulations permit, are added to the formulation to create a desired taste or aroma in a product for adult consumers. Reference here to “flavor” or “flavorant” includes both singular and multi-component flavors.

In one aspect the flavor is selected from the group consisting of extracts, for example liquorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, flavor enhancers, bitterness receptor site blockers, sensorial receptor site activators or stimulators, sugars and/or sugar substitutes (e.g. sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

In one aspect, the formulation further comprises an active agent. By “active agent” it is meant an agent which has a biological effect on a subject when the vapor is inhaled. The one or more active agents may be selected from nicotine, botanicals, and mixtures thereof. The one or more active agents may be of synthetic or natural origin. The active could be an extract from a botanical, such as from a plant in the tobacco family.

An example active is nicotine.

Thus in one aspect the present invention provides a vaporizable formulation comprising:

(i) one or more solvents,

(ii) less than about 1 wt % of a cooling agent based on the total weight of the vaporizable formulation, wherein the cooling agent volatilizes at a higher temperature than menthol at atmospheric pressure, and

(iii) an active agent

or

(i) one or more solvents,

(ii) less than about 12 wt % of a cooling agent based on the total weight of the vaporizable formulation, wherein the cooling agent volatilizes at a higher temperature than menthol at atmospheric pressure, and is a compound of formula (I) or salt and/or solvate thereof as defined herein, and

(iii) an active agent.

In one aspect the active agent is nicotine.

Thus in one aspect the present invention provides a vaporizable formulation comprising:

(i) one or more solvents,

(ii) less than about 1 wt % of a cooling agent based on the total weight of the vaporizable formulation, wherein the cooling agent volatilizes at a higher temperature than menthol at atmospheric pressure, and

(iii) nicotine

or

(i) one or more solvents,

(ii) less than about 12 wt % of a cooling agent based on the total weight of the vaporizable formulation, wherein the cooling agent volatilizes at a higher temperature than menthol at atmospheric pressure, and is a compound of formula (I) or a salt and/or solvate thereof as defined herein, and

(iii) nicotine.

Nicotine may be provided in any suitable amount depending on the desired dosage when inhaled by the user. In one aspect nicotine is present in an amount of no greater than about 6 wt % based on the total weight of the vaporizable formulation. In one aspect nicotine is present in an amount of from about 0.4 to about 6 wt % based on the total weight of the vaporizable formulation. In one aspect nicotine is present in an amount of from about 0.8 to about 6 wt % based on the total weight of the vaporizable formulation. In one aspect nicotine is present in an amount of from about 1 to about 6 wt % based on the total weight of the vaporizable formulation. In one aspect nicotine is present in an amount of from about 1.8 to about 6 wt % based on the total weight of the vaporizable formulation.

In one aspect nicotine is present in an amount of no greater than about 5 wt % based on the total weight of the vaporizable formulation. In one aspect nicotine is present in an amount of from about 0.4 to about 5 wt % based on the total weight of the vaporizable formulation. In one aspect nicotine is present in an amount of from about 0.8 to about 5 wt % based on the total weight of the vaporizable formulation. In one aspect nicotine is present in an amount of from about 1 to about 5 wt % based on the total weight of the vaporizable formulation. In one aspect nicotine is present in an amount of from about 1.8 to about 5 wt % based on the total weight of the vaporizable formulation.

In one aspect nicotine is present in an amount of no greater than about 4 wt % based on the total weight of the vaporizable formulation. In one aspect nicotine is present in an amount of from about 0.4 to about 4 wt % based on the total weight of the vaporizable formulation. In one aspect nicotine is present in an amount of from about 0.8 to about 4 wt % based on the total weight of the vaporizable formulation. In one aspect nicotine is present in an amount of from about 1 to about 4 wt % based on the total weight of the vaporizable formulation. In one aspect nicotine is present in an amount of from about 1.8 to about 4 wt % based on the total weight of the vaporizable formulation.

In one aspect nicotine is present in an amount of no greater than about 3 wt % based on the total weight of the vaporizable formulation. In one aspect nicotine is present in an amount of from about 0.4 to about 3 wt % based on the total weight of the vaporizable formulation. In one aspect nicotine is present in an amount of from about 0.8 to about 3 wt % based on the total weight of the vaporizable formulation. In one aspect nicotine is present in an amount of from about 1 to about 3 wt % based on the total weight of the vaporizable formulation. In one aspect nicotine is present in an amount of from about 1.8 to about 3 wt % based on the total weight of the e vaporizable formulation.

In one aspect nicotine is present in an amount of no greater than about 1.9 wt % based on the total weight of the vaporizable formulation. In one aspect nicotine is present in an amount of no greater than about 1.8 wt % based on the total weight of the vaporizable formulation. In one aspect nicotine is present in an amount of from about 0.4 to about 1.9 wt % based on the total weight of the vaporizable formulation. In one aspect nicotine is present in an amount of from about 0.4 to about 1.8 wt % based on the total weight of the vaporizable formulation. In one aspect nicotine is present in an amount of from about 0.5 to about 1.9 wt % based on the total weight of the vaporizable formulation. In one aspect nicotine is present in an amount of-from about 0.5 to about 1.8 wt % based on the total weight of the vaporizable formulation. In one aspect nicotine is present in an amount of from about 0.8 to about 1.9 wt % based on the total weight of the vaporizable formulation. In one aspect nicotine is present in an amount of from about 0.8 to about 1.8 wt % based on the total weight of the vaporizable formulation. In one aspect nicotine is present in an amount of from about 1 to about 1.9 wt % based on the total weight of the vaporizable formulation. In one aspect nicotine is present in an amount of from about 1 to about 1.8 wt % based on the total weight of the vaporizable formulation.

In one aspect nicotine is present in an amount of less than about 1.9 wt % based on the total weight of the vaporizable formulation. In one aspect nicotine is present in an amount of less than about 1.8 wt % based on the total weight of the vaporizable formulation. In one aspect nicotine is present in an amount of from about 0.4 to less than about 1.9 wt % based on the total weight of the vaporizable formulation. In one aspect nicotine is present in an amount of from about 0.4 to less than about 1.8 wt % based on the total weight of the vaporizable formulation. In one aspect nicotine is present in an amount of from about 0.5 to less than about 1.9 wt % based on the total weight of the vaporizable formulation. In one aspect nicotine is present in an amount of from about 0.5 to less than about 1.8 wt % based on the total weight of the vaporizable formulation. In one aspect nicotine is present in an amount of from about 0.8 to less than about 1.9 wt % based on the total weight of the vaporizable formulation. In one aspect nicotine is present in an amount of from about 0.8 to less than about 1.8 wt % based on the total weight of the vaporizable formulation. In one aspect nicotine is present in an amount of from about 1 to less than about 1.9 wt % based on the total weight of the vaporizable formulation. In one aspect nicotine is present in an amount of from about 1 to less than about 1.8 wt % based on the total weight of the vaporizable formulation.

In one aspect, the vaporizable formulation may contain one or acids. In some embodiments, the vaporizable formulation may contain one or more acids in addition to nicotine (as the active agent). In some embodiments, the one or more acids may be one or more organic acids. In some embodiments, the one or more acids may be one or more organic acids selected from the group consisting of benzoic acid, levulinic acid, malic acid, maleic acid, fumaric acid, citric acid, lactic acid, acetic acid, succinic acid, and mixtures thereof. When included in the formulation in combination with nicotine, the one or more acids may provide a formulation in which the nicotine is at least partially in protonated (such as monoprotonated and/or diprotonated) form.

Solvent

As discussed herein, the vaporizable formulation includes one or more solvents.

The one or more solvents may be any suitable solvents. In one aspect, the one or more solvents are selected from water, glycerol, propylene glycol, 1,3-propanediol and mixtures thereof. Other suitable solvents will be apparent to one skilled in the art.

The one or more solvents may be present in any suitable amount in the vaporizable formulation. In one aspect the one or more solvents is present in a total amount of at least 5 wt % based on the total weight of the vaporizable formulation. In one aspect the one or more solvents is present in a total amount of at least 10 wt % based on the total weight of the vaporizable formulation. In one aspect the one or more solvents is present in a total amount of at least 15 wt % based on the total weight of the vaporizable formulation. In one aspect the one or more solvents is present in a total amount of at least 20 wt % based on the total weight of the vaporizable formulation. In one aspect the one or more solvents is present in a total amount of at least 25 wt % based on the total weight of the vaporizable formulation. In one aspect the one or more solvents is present in a total amount of at least 30 wt % based on the total weight of the vaporizable formulation. In one aspect the one or more solvents is present in a total amount of at least 35 wt % based on the total weight of the vaporizable formulation. In one aspect the one or more solvents is present in a total amount of at least 40 wt % based on the total weight of the vaporizable formulation. In one aspect the one or more solvents is present in a total amount of at least 45 wt % based on the total weight of the vaporizable formulation. In one aspect the one or more solvents is present in a total amount of at least 50 wt % based on the total weight of the vaporizable formulation. In one aspect the one or more solvents is present in a total amount of at least 55 wt % based on the total weight of the vaporizable formulation. In one aspect the one or more solvents is present in a total amount of at least 60 wt % based on the total weight of the vaporizable formulation. In one aspect the one or more solvents is present in a total amount of at least 65 wt % based on the total weight of the vaporizable formulation. In one aspect the one or more solvents is present in a total amount of at least 70 wt % based on the total weight of the vaporizable formulation. In one aspect the one or more solvents is present in a total amount of at least 75 wt % based on the total weight of the vaporizable formulation. In one aspect the one or more solvents is present in a total amount of at least 80 wt % based on total weight of the vaporizable formulation. In one aspect the one or more solvents is present in a total amount of at least 85 wt % based on the total weight of the vaporizable formulation. In one aspect the one or more solvents is present in a total amount of at least 90 wt % based on the total weight of the vaporizable formulation.

In one aspect the one or more solvents is present in a total amount of from 5 to 99 wt % based on the vaporizable formulation. In one aspect the one or more solvents is present in a total amount of from 10 to 99 wt % based on the vaporizable formulation. In one aspect the one or more solvents is present in a total amount of from 15 to 99 wt % based on the vaporizable formulation. In one aspect the one or more solvents is present in a total amount of from 20 to 99 wt % based on the vaporizable formulation. In one aspect the one or more solvents is present in a total amount of from 25 to 99 wt % based on the vaporizable formulation. In one aspect the one or more solvents is present in a total amount of from 30 to 99 wt % based on the vaporizable formulation. In one aspect the one or more solvents is present in a total amount of from 35 to 99 wt % based on the vaporizable formulation. In one aspect the one or more solvents is present in a total amount of from 40 to 99 wt % based on the vaporizable formulation. In one aspect the one or more solvents is present in a total amount of from 45 to 99 wt % based on the vaporizable formulation. In one aspect the one or more solvents is present in a total amount of from 50 to 99 wt % based on the vaporizable formulation. In one aspect the one or more solvents is present in a total amount of from 55 to 99 wt % based on the vaporizable formulation. In one aspect the one or more solvents is present in a total amount of from 60 to 99 wt % based on the vaporizable formulation. In one aspect the one or more solvents is present in a total amount of from 65 to 99 wt % based on the vaporizable formulation. In one aspect the one or more solvents is present in a total amount of from 70 to 99 wt % based on the vaporizable formulation. In one aspect the one or more solvents is present in a total amount of from 75 to 99 wt % based on the vaporizable formulation. In one aspect the one or more solvents is present in a total amount of from 80 to 99 wt % based on the vaporizable formulation. In one aspect the one or more solvents is present in a total amount of from 85 to 99 wt % based on the vaporizable formulation. In one aspect the one or more solvents is present in a total amount of from 90 to 99 wt % based on the vaporizable formulation.

In one aspect the one or more solvents is present in a total amount of from 5 to 95 wt % based on the vaporizable formulation. In one aspect the one or more solvents is present in a total amount of from 10 to 95 wt % based on the vaporizable formulation. In one aspect the one or more solvents is present in a total amount of from 15 to 95 wt % based on the vaporizable formulation. In one aspect the one or more solvents is present in a total amount of from 20 to 95 wt % based on the vaporizable formulation. In one aspect the one or more solvents is present in a total amount of from 25 to 95 wt % based on the vaporizable formulation. In one aspect the one or more solvents is present in a total amount of from 30 to 95 wt % based on the vaporizable formulation. In one aspect the one or more solvents is present in a total amount of from 35 to 95 wt % based on the vaporizable formulation. In one aspect the one or more solvents is present in a total amount of from 40 to 95 wt % based on the vaporizable formulation. In one aspect the one or more solvents is present in a total amount of from 45 to 95 wt % based on the vaporizable formulation. In one aspect the one or more solvents is present in a total amount of from 50 to 95 wt % based on the vaporizable formulation. In one aspect the one or more solvents is present in a total amount of from 55 to 95 wt % based on the vaporizable formulation. In one aspect the one or more solvents is present in a total amount of from 60 to 95 wt % based on the vaporizable formulation. In one aspect the one or more solvents is present in a total amount of from 65 to 95 wt % based on the vaporizable formulation. In one aspect the one or more solvents is present in a total amount of from 70 to 95 wt % based on the vaporizable formulation. In one aspect the one or more solvents is present in a total amount of from 75 to 95 wt % based on the vaporizable formulation. In one aspect the one or more solvents is present in a total amount of from 80 to 95 wt % based on the vaporizable formulation. In one aspect the one or more solvents is present in a total amount of from 85 to 95 wt % based on the vaporizable formulation. In one aspect the one or more solvents is present in a total amount of from 90 to 95 wt % based on the vaporizable formulation.

In one aspect the one or more solvents is present in a total amount of from 5 to 90 wt % based on the vaporizable formulation. In one aspect the one or more solvents is present in a total amount of from 10 to 90 wt % based on the vaporizable formulation. In one aspect the one or more solvents is present in a total amount of from 15 to 90 wt % based on the vaporizable formulation. In one aspect the one or more solvents is present in a total amount of from 20 to 90 wt % based on the vaporizable formulation. In one aspect the one or more solvents is present in a total amount of from 25 to 90 wt % based on the vaporizable formulation. In one aspect the one or more solvents is present in a total amount of from 30 to 90 wt % based on the vaporizable formulation. In one aspect the one or more solvents is present in a total amount of from 35 to 90 wt % based on the vaporizable formulation. In one aspect the one or more solvents is present in a total amount of from 40 to 90 wt % based on the vaporizable formulation. In one aspect the one or more solvents is present in a total amount of from 45 to 90 wt % based on the vaporizable formulation. In one aspect the one or more solvents is present in a total amount of from 50 to 90 wt % based on the vaporizable formulation. In one aspect the one or more solvents is present in a total amount of from 55 to 90 wt % based on the vaporizable formulation. In one aspect the one or more solvents is present in a total amount of from 60 to 90 wt % based on the vaporizable formulation. In one aspect the one or more solvents is present in a total amount of from 65 to 90 wt % based on the vaporizable formulation. In one aspect the one or more solvents is present in a total amount of from 70 to 90 wt % based on the vaporizable formulation. In one aspect the one or more solvents is present in a total amount of from 75 to 90 wt % based on the vaporizable formulation. In one aspect the one or more solvents is present in a total amount of from 80 to 90 wt % based on the vaporizable formulation. In one aspect the one or more solvents is present in a total amount of from 85 to 90 wt % based on the vaporizable formulation.

As discussed herein, in one aspect the solvent is at least glycerol. The glycerol may be present in any suitable amount in the vaporizable formulation. In one aspect the glycerol is present in a total amount of at least 10 wt % based on the-vaporizable formulation. In one aspect the glycerol is present in a total amount of at least 20 wt % based on the vaporizable formulation. In one aspect the glycerol is present in a total amount of at least 30 wt % based on the vaporizable formulation. In one aspect the glycerol is present in a total amount of at least 35 wt % based on the vaporizable formulation. In one aspect the glycerol is present in a total amount of at least 40 wt % based on the vaporizable formulation. In one aspect the glycerol is present in a total amount of at least 45 wt % based on the vaporizable formulation. In one aspect the glycerol is present in a total amount of at least 50 wt % based on the vaporizable formulation. In one aspect the glycerol is present in a total amount of at least 55 wt % based on the vaporizable formulation. In one aspect the glycerol is present in a total amount of at least 60 wt % based on the formulation.

In one aspect the glycerol is present in a total amount of from 10 to 60 wt % based on the formulation. In one aspect the glycerol is present in a total amount of from 20 to 60 wt % based on the formulation. In one aspect the glycerol is present in a total amount of from 25 to 60 wt % based on the formulation. In one aspect the glycerol is present in a total amount of from 30 to 60 wt % based on the vaporizable formulation. In one aspect the glycerol is present in a total amount of from 35 to 60 wt % based on the vaporizable formulation. In one aspect the glycerol is present in a total amount of from 40 to 60 wt % based on the vaporizable formulation. In one aspect the glycerol is present in a total amount of from 45 to 60 wt % based on the vaporizable formulation.

As discussed herein, in one aspect the solvent is at least propylene glycol. The propylene glycol may be present in any suitable amount in the vaporizable formulation. In one aspect the propylene glycol is present in a total amount of at least 10 wt % based on the vaporizable formulation. In one aspect the propylene glycol is present in a total amount of at least 15 wt % based on the vaporizable formulation. In one aspect the propylene glycol is present in a total amount of at least 20 wt % based on the vaporizable formulation. In one aspect the propylene glycol is present in a total amount of at least 25 wt % based on the vaporizable formulation. In one aspect the propylene glycol is present in a total amount of at least 30 wt % based on the vaporizable formulation. In one aspect the propylene glycol is present in a total amount of at least 35 wt % based on the vaporizable formulation. In one aspect the propylene glycol is present in a total amount of at least 40 wt % based on the vaporizable formulation.

In one aspect the propylene glycol is present in a total amount of from 10 to 40 wt % based on the vaporizable formulation. In one aspect the propylene glycol is present in a total amount of from 15 to 40 wt % based on the vaporizable formulation. In one aspect the propylene glycol is present in a total amount of from 20 to 40 wt % based on the vaporizable formulation. In one aspect the propylene glycol is present in a total amount of from 25 to 40 wt % based on the vaporizable formulation. In one aspect the propylene glycol is present in a total amount of from 30 to 40 wt % based on the vaporizable formulation. In one aspect the propylene glycol is present in a total amount of from 32 to 40 wt % based on the vaporizable formulation. In one aspect the propylene glycol is present in a total amount of from 35 to 40 wt % based on the vaporizable formulation.

As discussed herein, the one or more solvents may include water. In one aspect the water is present in a total amount of from 0 to 25 wt % based on the vaporizable formulation. This amount includes 0% water based on the vaporizable formulation and therefore the vaporizable formulation may contain water or may not contain water.

In one aspect the vaporizable formulation contains water. The water may be present in any suitable amount in the vaporizable formulation. In one aspect water is present in a total amount of from 0 to 24 wt % based on the vaporizable formulation. In one aspect water is present in a total amount of from 0 to 22 wt % based on the vaporizable formulation. In one aspect water is present in a total amount of from 0 to 20 wt % based on the vaporizable formulation. In one aspect water is present in a total amount of from 0 to 19 wt % based on the vaporizable formulation. In one aspect water is present in a total amount of from 0 to 18 wt % based on the vaporizable formulation. In one aspect water is present in a total amount of from 0 to 17 wt % based on the vaporizable formulation. In one aspect water is present in a total amount of from 0 to 16 wt % based on the vaporizable formulation. In one aspect water is present in a total amount of from 0 to 15 wt % based on the vaporizable formulation.

In an alternative embodiment, the solvent comprises substantially no water. In this regard, it is noted that some formulations may be hygroscopic and as such water ingress into the formulation cannot be ruled out. Accordingly, by “substantially no water” it is meant that the amount of water present is less than 0.1 wt % based on the vaporizable formulation.

Process

As discussed herein, the present invention provides a process for forming a vapor, the process comprising vaporizing a vaporizable formulation comprising (i) one or more solvents and (ii) less than about 1 wt % of a cooling agent based on the total weight of the vaporizable formulation, wherein the cooling agent volatilizes at a higher temperature than menthol at atmospheric pressure or less than about 12 wt % of a cooling agent based on the total weight of the vaporizable formulation, wherein the cooling agent volatilizes at a higher temperature than menthol at atmospheric pressure and is a compound of formula (I) or a salt and/or solvate thereof as defined herein.

In one aspect, the vapor is formed by heating the vaporizable formulation to a temperature above about 100° C. In one aspect, the vapor is formed by heating the vaporizable formulation to a temperature above about 110° C. In one aspect, the vapor is formed by heating the vaporizable formulation to a temperature above about 120° C.

In another aspect, the vapor is formed by a process performed at a temperature below about 100° C. In one aspect, the vapor is formed by a process performed at a temperature below about 90° C. In one aspect, the vapor is formed by a process performed at a temperature below about 80° C.

In one aspect, the vapor is formed by applying ultrasonic energy to the vaporizable formulation.

Further Aspects

The vaporizable formulation may be contained or delivered by any means. In one aspect the present invention provides a contained vaporizable formulation comprising:

(a) a container; and

(b) a vaporizable formulation as defined hereinabove.

The container may be any suitable container, for example to allow for the storage or delivery of the solution. In one aspect the container is configured for engagement with an electronic vapor provision system. The container may be a bottle. The container may be configured to become fluidly in communication with an electronic vapor provision system so that solution may be delivered to the electronic vapor provision system. As described above, the present disclosure relates to container which may be used in an electronic vapor provision system, such as an e-cigarette. Throughout the following description the term “e-cigarette” is used; however, this term may be used interchangeably with electronic vapor provision system.

As discussed herein, the container of the present invention is typically provided for the delivery of vaporizable formulation to or within an e-cigarette. The vaporizable formulation may be held within an e-cigarette or may be sold as a separate container for subsequent use with or in an e-cigarette. As understood by one skilled in the art, e-cigarettes may contain a unit known as a detachable cartomizer which typically comprises a reservoir of vaporizable formulation, a wick material and a device for vaporizing the vaporizable formulation. In some e-cigarettes, the cartomizer is part of a single-piece device and is not detachable. In one aspect the container is a cartomizer or is part of a cartomizer. In one aspect the container is not a cartomizer or part of a cartomizer and is a container, such as a tank, which may be used to deliver vaporizable formulation to or within an e-cigarette.

In one aspect the container is part of an electronic vapor provision system, such as an e-cigarette. Therefore in a further aspect the present invention provides an electronic vapor provision system comprising:

(a) a vaporizable for vaporizing the formulation for inhalation by a user of the electronic vapor provision system;

(b) a power supply comprising a cell or battery for supplying power to the vaporizable; and

(c) a vaporizable formulation as described hereinabove.

In addition to the vaporizable formulations of the present invention and to systems such as containers and electronic aerosol provision systems containing the same, the present invention provides use of a cooling agent for extending the shelf-life of a vaporizable formulation, wherein the cooling agent volatilizes at a higher temperature than menthol at atmospheric pressure.

Shelf life is typically the length of time that a consumer product may be stored without becoming unfit for use, consumption or sale. By the expression “extending the shelf-life” is meant that the cooling agent allows the vaporizable formulation to be stored for a longer period of time without chemical decomposition of its components when compared to a similar formulation including menthol. Typical storage conditions include ambient temperature and pressure.

### EXAMPLES

The invention will now be described with reference to the following non-limiting examples.

Nine cooling agents were used in the Examples. These compounds are shown in Table 1 below along with a chemical name, a trade name, approximate boiling point and approximate vapor pressure. Given that the compounds are known, their boiling points and vapor pressure have been obtained from the literature.

TABLE 1
Cooling Agent Compounds with Boiling Points and Vapor Pressures.

| | | | Boiling | |
|---|---|---|---|---|
| | | | point (° C.) | |
| | | | @ 760 | Vapor |
| | | | mmHg | pressure |
| | Trade | | unless | (mmHg) |
| Chemical Name | Name | Chemical Structure | stated | @ 25° C. |
| (1S,2R,5S)-N-ethyl-5- methyl-2-(propan-2- yl)cyclohexane- carboxamide | WS-3 | 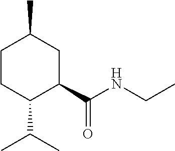 | 340.55 | 8.50E−5 |
| ethyl-2-[[(1R,2S,5R)-5- methyl-2-propan-2- ylcyclohexanecarbonyl] amino]acetate | WS-5 | 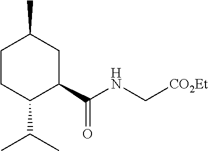 [SMILES: CCOC(=O)CNC(=O)[C@@H]1C[C@H](C)CC[C@H]1C(C)C] | 151.00 (@ 2.00 mmHg) | unknown |
| (1R,2S,5R)-N-(4- methoxyphenyl-p- menthanecarboxamide | WS-12 | 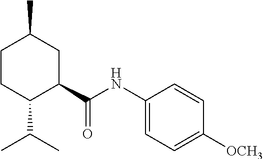 [SMILES: COc1ccc(NC(=O)[C@@H]2C[C@H](C)CC[C@H]2C(C)C)cc1] | 447.8 ± 28.0 | 3.25E−8 |
| N,2,3-trimethyl-2- propan-2-ylbutanamide | WS-23 | 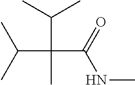 | 233.00-234.00 | 5.73E2 |
| (1R,2S,5R)-N-(2- (pyridin-2-yl)ethyl) menthylcarboxamide | Evercool ® 190 | 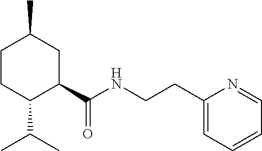 [SMILES: CC(C)[C@@H]1CC[C@@H](C)C[C@H]1C(=O)NCCc1ccccn1] | 461.6 ± 24.0 | 1.06E−8 |
| (−)-menthone 1,2-glycerol ketal | Frescolat ® MGA | 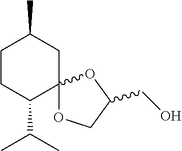 [SMILES: CC(C)[C@@H]1CC[C@@H](C)CC12OCC(CO)O2] | 138-142 | 2.15E−5 |
| (−)-menthyl lactate | Frescolat ® ML | 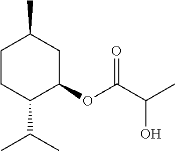 [SMILES: CC(O)C(=O)O[C@@H]1C[C@H](C)CC[C@H]1C(C)C] | 304.0 ± 15.0 | 8.58E−5 |
| 3-((−)-menthoxy) propane-1,2- diol | Coolact ® 10 | 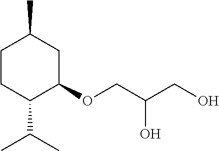 [SMILES: CC(C)[C@@H]1CC[C@@H](C)C[C@H]1OCC(O)CO] | 362.8 ± 22.0 | 9.88E−7 |
| (−)-menthyl succinate | Monomenthyl succinate | 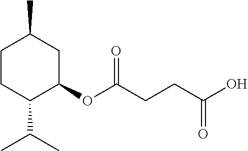 [SMILES: CC(C)[C@@H]1CC[C@@H](C)C[C@H]1OC(=O)CCC(=O)O] | 374.35 | 1.23E−6 |

Analytical Testing

Thermogravimetric Analysis

Prior to panel testing, the cooling agents were subjected to thermogravimetric analysis (TGA) to understand their behavior as they are heated. As is known in the art, TGA is an analytical method which can monitor the mass loss of a sample as it is heated to temperatures of up to 1000° C. and higher.

All measurements were carried out on a Perkin Elmer STA6000 TGA thermogravimetric analyzer. For solid samples, a spatula was used to transfer 5-20 mg of material into the crucible of the analyzer. Oils were introduced to the instrument by transferring 5-20 mg using a Pasteur pipette onto a small portion of Cambridge filter pad which was placed in the crucible. All samples were heated from 30° C. to 600° C. at a constant rate of 5° C./min. This was carried out for each compound twice in air and twice in nitrogen. For a small number of samples, the run was stopped at 500-600° C. when full mass loss had been observed. For comparative purposes, menthol was also analyzed.

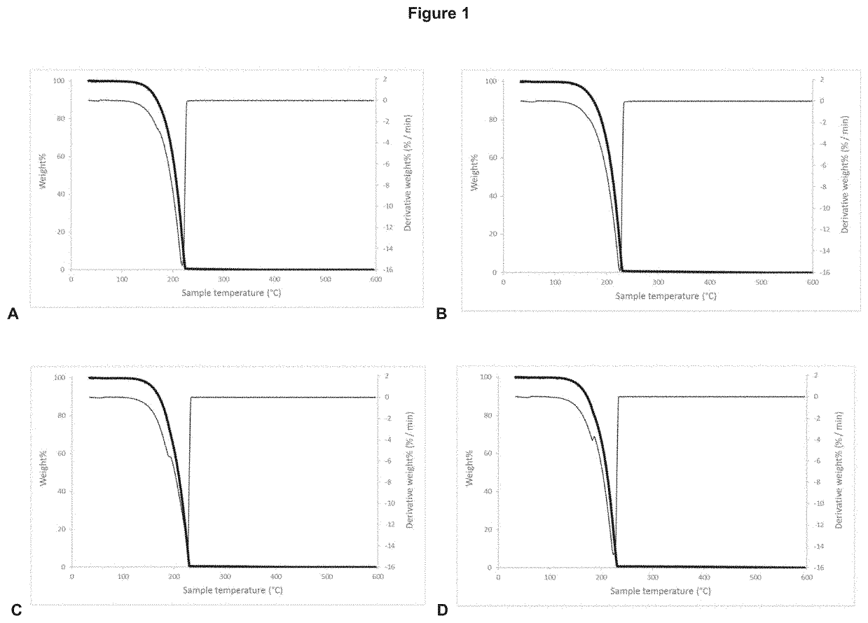

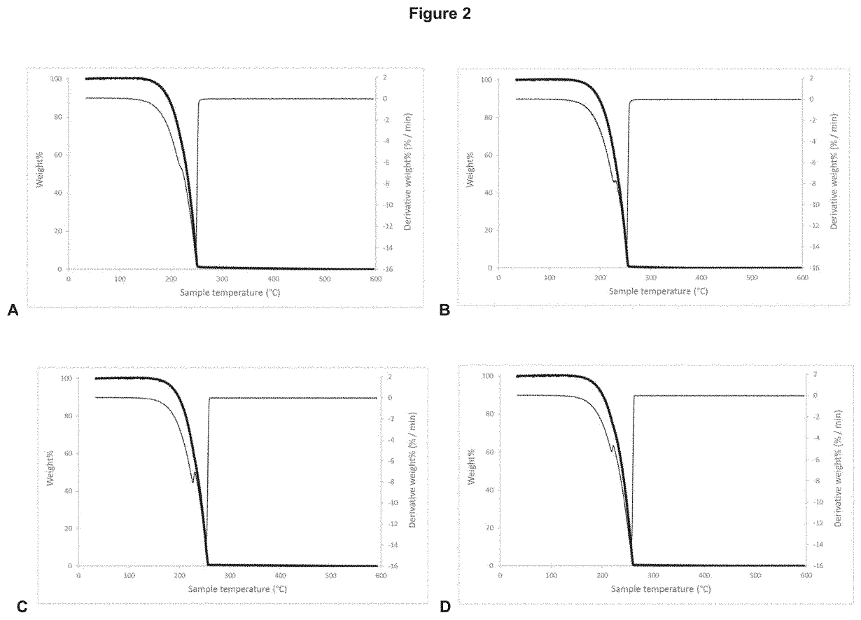

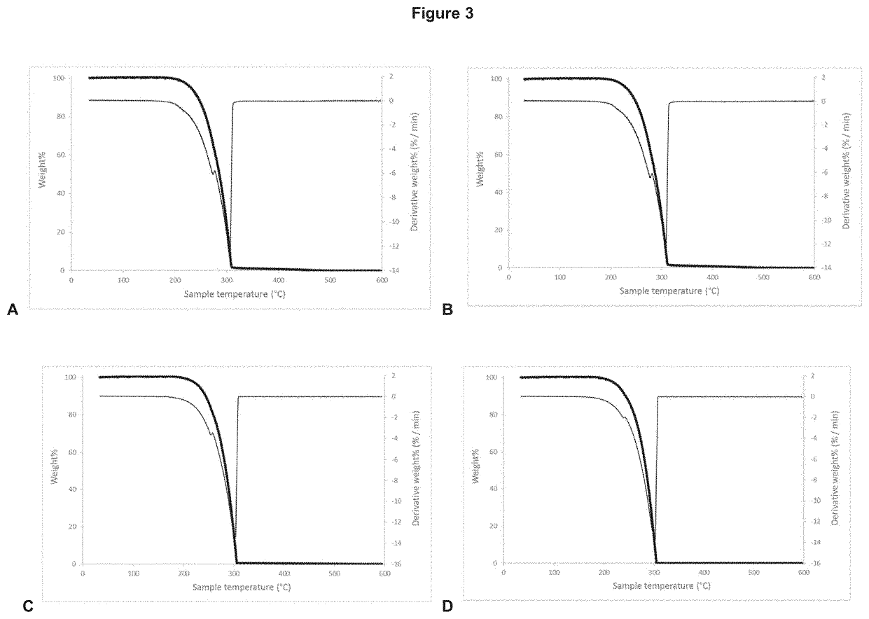

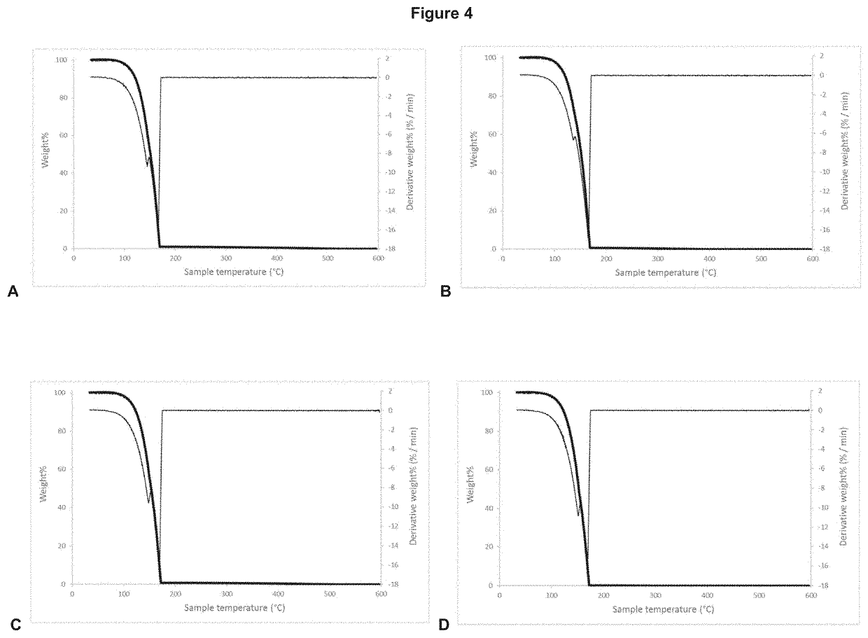

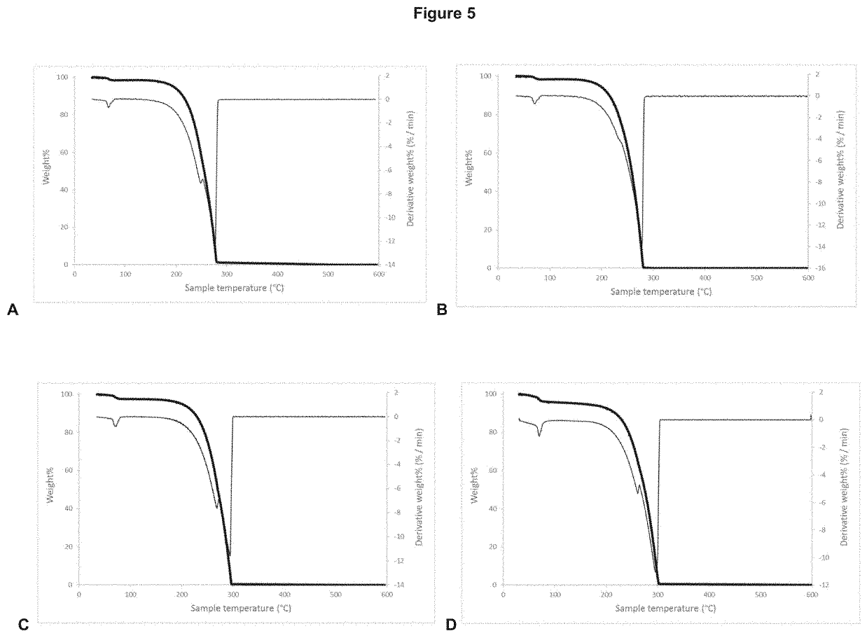

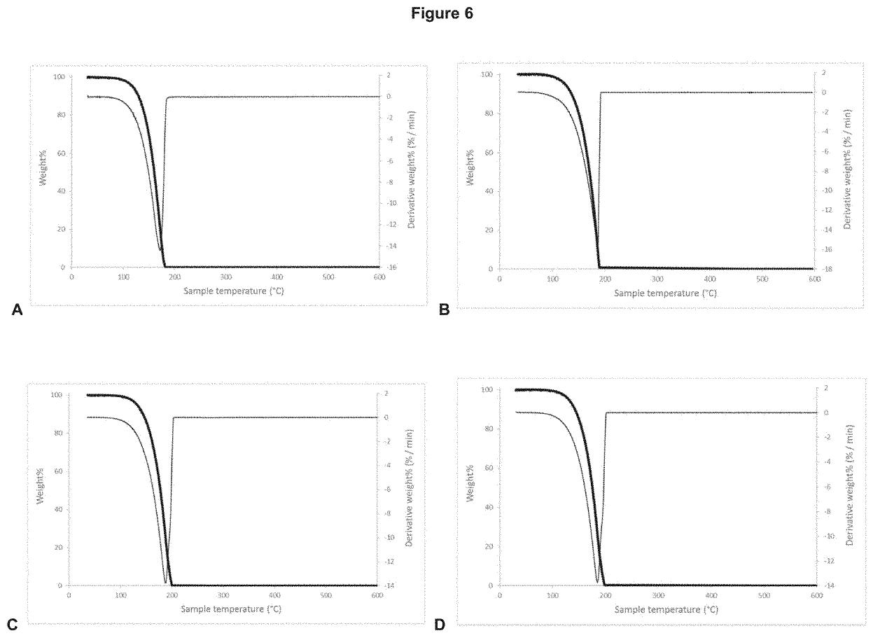

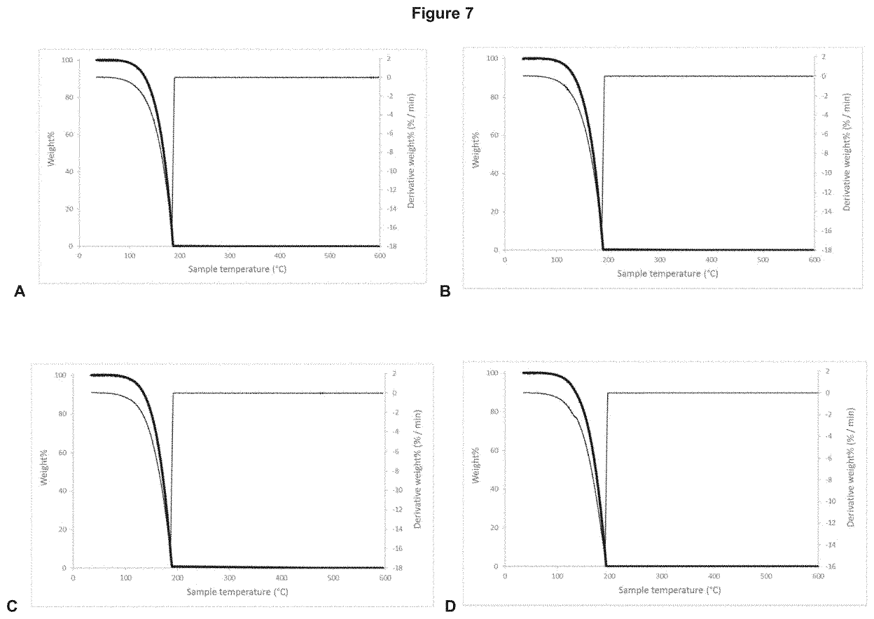

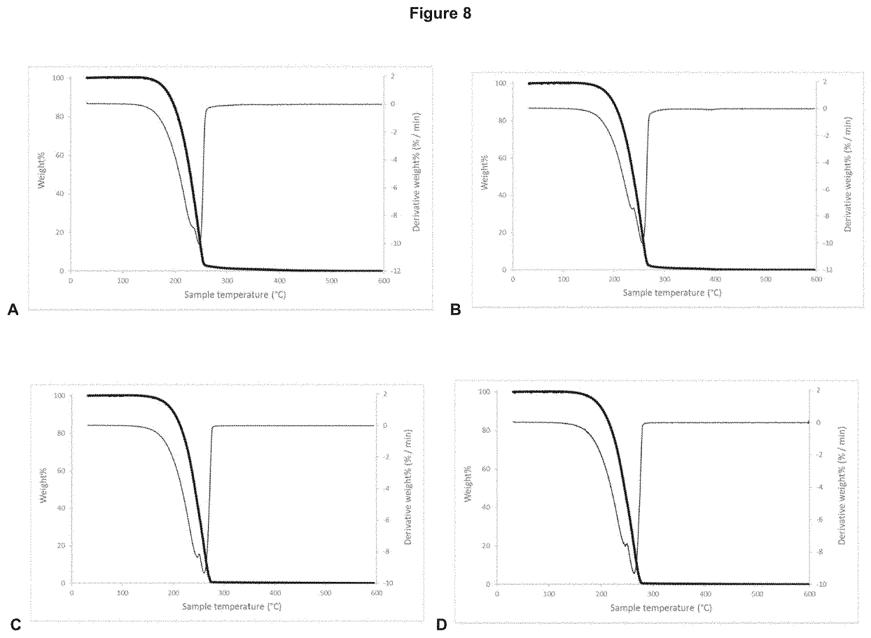

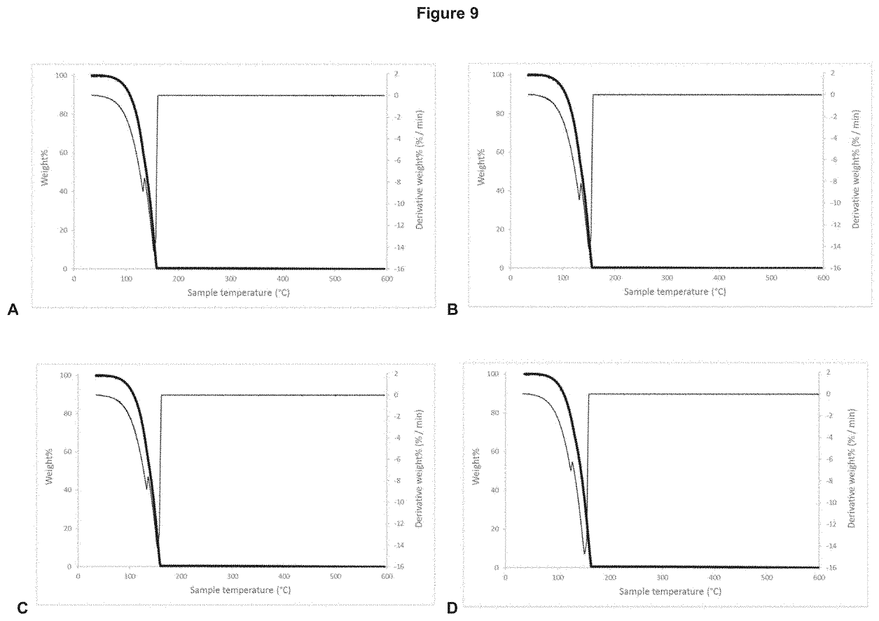

The TGA traces for each compound (rep 1 and rep 2 in air; and rep 1 and rep 2 in nitrogen) are shown in FIGS. 1 to 9 (A-D). The onset temperature of mass loss for each compound was determined by visual inspection of the TGA traces on the computer software and the results are summarized in Table 2. Evercool™ 190 showed more than one mass loss event but the mass loss at approximately 65-75° C. was confirmed as due to the presence of water in the sample.

TABLE 2
Summary of Thermogravimetric Analyzer
Traces for Cooling Agent Compounds.

| | Tₚₑₐₖ of mass loss (° C.) | | | |
|---|---|---|---|---|
| | Air | Air | Nitrogen | Nitrogen |
| Compound | (rep 1) | (rep 2) | (rep 1) | (rep 2) |
| WS-3 | 127 | 132 | 132 | 128 |
| WS-5 | 149 | 151 | 154 | 158 |
| WS-12 | 202 | 203 | 199 | 197 |
| WS-23 | 88 | 88 | 90 | 86 |
| Evercool ™ 190 | 161 | 164 | 169 | 174 |
| Frescolat ® MGA | 93 | 85 | 102 | 94 |
| Frescolat ® ML | 86 | 88 | 86 | 91 |
| Coolact ® 10 | 119 | 112 | 115 | 119 |
| Monomenthyl succinate | 144 | 148 | 151 | 151 |
| Menthol | 53 | 55 | 52 | 55 |

It can be seen from these results that good repeatability of the experiments was achieved and there were insignificant differences between TGA traces in air and nitrogen. The latter suggests the compounds are not susceptible to thermal oxidation at the temperatures at which mass loss was observed.

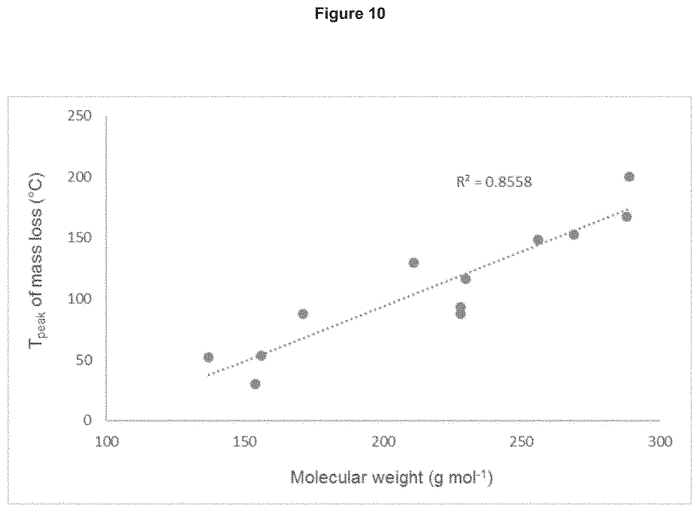

These results also show that unlike menthol, mass loss for all of the cooling agents tested occurred at a temperature above 85° C. As this mass loss was generally positively correlated with molecular weight (see FIG. 10), it can be attributed to volatilization/vaporization rather than decomposition. All of the compounds tested are therefore less volatile than menthol and can be said to volatilize at a temperature higher than menthol at atmospheric pressure. The reduced volatility of the cooling agents is advantageous because it means that they are more stable under storage than menthol.

### Example 1

Observational Testing

10 volunteers were asked to use each cooling agent sample in a Vype eTank Pro (an electronic cigarette device). All cooling agents were used at a concentration of 0.18% except for menthol which in one sample was used at the current commercial concentration of 1.235%. Each of the menthol samples were tested for comparative purposes.

Each volunteer was asked to assess the cooling intensity on an objective basis (i.e. not considering personal preference) and a subjective basis. The objective assessment required the volunteer to rate the overall perceived cooling intensity from 0 to 10, where 0 was no cooling perceivable and 10 was very strong cooling sensation, and to place the cooling sensation, e.g. in the mouth, throat etc. The subjective assessment required the cooling intensity to be ranked as (1) much too low, (2) slightly too low, (3) just right, (4) slightly too high or (5) much too high.

Results

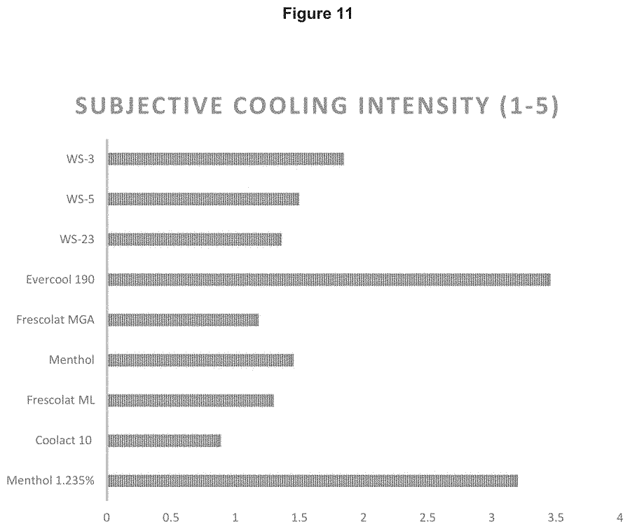

The results from the objective and subjective assessment of the volunteers are shown in Table 3 below and FIG. 11.

TABLE 3
Average Cooling Intensity of Cooling Agent Compounds.

| | Average Cooling |
|---|---|
| Cooling Agent | Intensity (0-10) |
| WS-3 | 3.045 |
| WS-5 | 2.5454 |
| WS-23 | 2.1818 |
| Evercool ® 190 | 7.1 |
| Frescolat MGA | 1 |
| Frescolat ML | 2 |
| Coolact 10 | 0.75 |
| Menthol (control) | 2.1 |
| Menthol (1.235%) | 8 |

The results show that some compounds provide a more cooling sensation at 0.18% than menthol at the same concentration. In particular, Evercool® 190 at 0.18% provides a cooling sensation of 7.1/10 compared to menthol at 1.235% of 8/10.

As cooling sensation delivery was generally more favorable for the non-menthol cooling agents compared with the menthol formulation, the cooling agents can be included in the vaporizable formulation of the present invention at much lower concentrations than menthol, and yet still deliver a cooling sensation when vaporized by the user. As shown by the thermogravimetric analysis above, the cooling agents also result in improved storage stability compared to menthol formulations as mass loss measured by TGA occurs at much higher temperatures. Finally, the results show that the cooling agents deliver a different cooling sensation to menthol. The area of delivery is mainly the mouth and the back of the throat, whilst there is minimal delivery on the tongue. This avoids the drying out of the mouth as experienced with menthol.

### Example 2

A minimum of 4 volunteers were asked to test each of the following e-liquid samples in an eTank Pro (an electronic cigarette device):
WS-3 at 2.5 wt % in glycerol/propylene glycol;
WS-3 at 5 wt % in glycerol/propylene glycol; and
WS-3 at 10 wt % in glycerol/propylene glycol.

WS-3 is a compound of formula (I) and is known by the chemical name of (1S,2R,5S)-N-ethyl-5-methyl-2-(propan-2-yl)cyclohexanecarboxamide.

Each volunteer was asked to assess the cooling intensity on an objective basis (i.e. not considering personal preference) and a subjective basis in the same manner as Example 1. The objective assessment required the volunteer to rate the overall perceived cooling intensity from 0 to 10, where 0 was no cooling perceivable and 10 was very strong cooling sensation, and to place the cooling sensation, e.g. in the mouth, throat etc. The subjective assessment required the cooling intensity to be ranked as (1) much too low, (2) slightly too low, (3) just right, (4) slightly too high or (5) much too high.

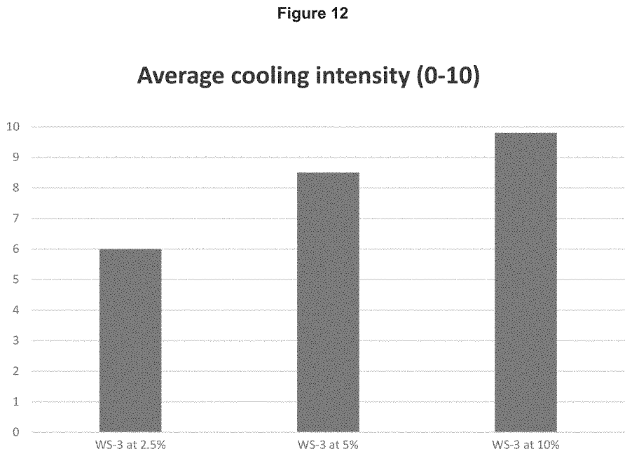

The results from the objective and subjective assessment of the volunteers are shown in Table 4 below and FIG. 12.

TABLE 4
Average Cooling Intensity of various WS-3 concentrations.

| | |
|---|---|
| Concentration of | Average Cooling |
| WS-3 | Intensity (0-10) |

| | | |
|---|---|---|
| 2.5 | wt % | 6 |
| 5 | wt % | 8.5 |
| 10 | wt % | 9.8 |

These results show that WS-3 cooling intensity increases as the concentration increases and that this compound can be included in an amount of 10 wt % whilst delivering a cooling sensory experience to the user. From the comments of the volunteers, it was also confirmed that the location of the cooling sensation is different to menthol even at higher concentrations. The area of delivery was mainly at the back of the throat with minimal delivery on the tongue. This sensation was also long-lasting and would be particularly beneficial in those markets that actively seek “high-menthol” or a strong cooling sensation.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. A vaporizable formulation comprising:
(i) one or more solvents; and
(ii) less than about 1 wt % of a cooling agent based on the weight of the vaporizable formulation;
wherein the cooling agent volatilizes at a higher temperature than menthol at atmospheric pressure.

2. The vaporizable formulation according to claim 1, wherein the formulation comprises about 0.001 to about 0.9 wt % of the cooling agent, based on the weight of the vaporizable formulation.

3. The vaporizable formulation according to claim 2, wherein the formulation comprises about 0.005 to about 0.75 wt % of the cooling agent, based on the weight of the vaporizable formulation.

4. The vaporizable formulation according to claim 3, wherein the formulation comprises about 0.01 to about 0.5 wt % of the cooling agent, based on the weight of the vaporizable formulation.

5. The vaporizable formulation according to claim 1, wherein the cooling agent volatilizes at a temperature of above 212° C. at atmospheric pressure.

6. The vaporizable formulation according to claim 5, wherein the cooling agent volatilizes at a temperature in the range of about 230° C. to about 500° C. at atmospheric pressure.

7. The vaporizable formulation according to claim 1, wherein the cooling agent has a vapor pressure lower than the vapor pressure of menthol at 25° C.

8. The vaporizable formulation according to claim 7, wherein the cooling agent has a vapor pressure lower than 0.008 mmHg at 25° C.

9. The vaporizable formulation according to claim 1, wherein the cooling agent is a compound of formula (I) or a salt and/or solvate thereof: 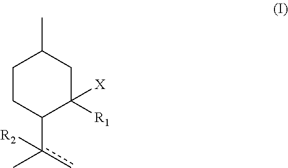
wherein X is hydrogen or OR′, wherein R′ is an alkyl group or an alkenyl group which may be taken together with R₁ to form a three to five-membered heterocycyl group, wherein the heterocycyl group is optionally substituted by one or more substituents selected from OH, O-alkyl, alkyl-OH, alkyl-O-alkyl, NH₂, NH-alkyl, N-(alkyl)₂, NO₂ and CN; and wherein R₁ and R₂ are each independently selected from OH, ORₐ, C(O)NR_{b}R_{c} and C(O)OR_{b}R_{c}; with the proviso that when R₁ is OH the compound of formula (I) is not menthol; and when the double bond is present, R₂ is absent;
wherein Rₐ is an alkyl group, an alkenyl group, a C(O)R_{f} group, or a C(O)-alkyl-C(O)R_{f} group wherein the alkyl groups and alkenyl groups are optionally substituted by one or more substituents selected from OH, O-alkyl, NH₂, NH-alkyl, N-(alkyl)₂, NO₂ and CN; and wherein R_{f} is an alkyl group, an alkenyl group, OH, O-alkyl, NH₂, NH-alkyl or N-(alkyl)₂, wherein the alkyl groups and alkenyl groups are optionally substituted by one or more substituents selected from OH, O-alkyl, NH₂, NH-alkyl, N-(alkyl)₂, NO₂ and CN;
wherein R_{b} and R_{c} are each independently hydrogen, an alkyl group, an alkenyl group, an aryl group, an aralkyl group, a heteroaryl group, or a heteroaralkyl group, wherein the alkyl groups, alkenyl groups, aryl groups and heteroaryl groups are optionally substituted by one or more substituents selected from OH, O-alkyl, NH₂, NH-alkyl, N-(alkyl)₂, NO₂, CN and C(O)R_{f}.

10. The vaporizable formulation according to claim 1, wherein the cooling agent is N,2,3-trimethyl-2-propan-2-ylbutanamide.

11. A vaporizable formulation comprising:
(i) one or more solvents; and
(ii) less than about 12 wt % of a cooling agent based on the weight of the vaporizable formulation;
wherein the cooling agent volatilizes at a higher temperature than menthol at atmospheric pressure and is a compound of formula (I) or a salt and/or solvate thereof: 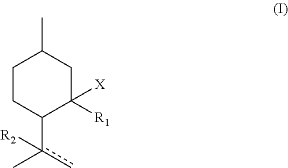
wherein X is hydrogen or OR′, wherein R′ is an alkyl group or an alkenyl group which may be taken together with R₁ to form a three to five-membered heterocycyl group, wherein the heterocycyl group is optionally substituted by one or more substituents selected from OH, O-alkyl, alkyl-OH, alkyl-O-alkyl, NH₂, NH-alkyl, N-(alkyl)₂, NO₂ and CN; and wherein R₁ and R₂ are each independently selected from OH, ORₐ, C(O)NR_{b}R_{c} and C(O)OR_{b}R_{c}; with the proviso that when R₁ is OH the compound of formula (I) is not menthol; and when the double bond is present, R₂ is absent;
wherein Rₐ is an alkyl group, an alkenyl group, a C(O)R_{f} group, or a C(O)-alkyl-C(O)R_{f} group wherein the alkyl groups and alkenyl groups are optionally substituted by one or more substituents selected from OH, O-alkyl, NH₂, NH-alkyl, N-(alkyl)₂, NO₂ and CN; and wherein R_{f} is an alkyl group, an alkenyl group, OH, O-alkyl, NH₂, NH-alkyl or N-(alkyl)₂, wherein the alkyl groups and alkenyl groups are optionally substituted by one or more substituents selected from OH, O-alkyl, NH₂, NH-alkyl, N-(alkyl)₂, NO₂ and CN;
wherein R_{b} and R_{c} are each independently hydrogen, an alkyl group, an alkenyl group, an aryl group, an aralkyl group, a heteroaryl group, or a heteroaralkyl group, wherein the alkyl groups, alkenyl groups, aryl groups and heteroaryl groups are optionally substituted by one or more substituents selected from OH, O-alkyl, NH₂, NH-alkyl, N-(alkyl)₂, NO₂, CN and C(O)R_{f}.

12. The vaporizable formulation according to claim 11, wherein the formulation comprises about 0.001 to about 11 wt % of the cooling agent, based on the weight of the vaporisable formulation.

13. The vaporizable formulation according to claim 12, wherein the formulation comprises about 0.005 to about 10 wt % of the cooling agent, based on the weight of the vaporisable formulation.

14. The vaporizable formulation according to claim 11, wherein the cooling agent is selected from the group consisting of N-ethyl-5-methyl-2-(propan-2-yl)cyclohexanecarboxamide, ethyl-2-[[5-methyl-2-propan-2-ylcyclohexanecarbonyl]amino] acetate, N-(4-methoxyphenyl-p-menthanecarboxamide, N-(2-(pyridin-2-yl)ethyl)menthylcarboxamide, menthone 1,2-glycerol ketal, menthyl lactate, 3-(menthoxy)propane-1,2-diol, and menthyl succinate.

15. The vaporizable formulation according to claim 11, wherein the one or more solvents are selected from water, glycerol, propylene glycol and mixtures thereof.

16. (canceled)

17. The vaporizable formulation according to claim 11, wherein the formulation further comprises an active agent.

18. The vaporizable formulation according to claim 17, wherein the active agent is nicotine.

19-23. (canceled)

24. The vaporizable formulation according to claim 1, wherein the cooling agent is selected from the group consisting of N-ethyl-5-methyl-2-(propan-2-yl)cyclohexanecarboxamide, ethyl-2-[[5-methyl-2-propan-2-ylcyclohexanecarbonyl]amino]acetate, N-(4-methoxyphenyl-p-menthanecarboxamide, N-(2-(pyridin-2-yl)ethyl)menthylcarboxamide, menthone 1,2-glycerol ketal, menthyl lactate, 3-(menthoxy)propane-1,2-diol, and menthyl succinate.

25. The vaporizable formulation according to claim 1, wherein the one or more solvents are selected from water, glycerol, propylene glycol and mixtures thereof.

26. A vaporizable formulation comprising:
(i) one or more solvents;
(ii) less than about 12 wt % of a cooling agent based on the weight of the vaporizable formulation; and
(iii) an active agent comprising nicotine;
wherein the cooling agent volatilizes at a higher temperature than menthol at atmospheric pressure;
wherein the cooling agent is a compound of formula (I) or a salt and/or solvate thereof: 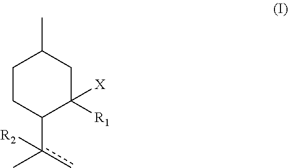
wherein X is hydrogen or OR′, wherein R′ is an alkyl group or an alkenyl group which may be taken together with R₁ to form a three to five-membered heterocycyl group, wherein the heterocycyl group is optionally substituted by one or more substituents selected from OH, O-alkyl, alkyl-OH, alkyl-O-alkyl, NH₂, NH-alkyl, N-(alkyl)₂, NO₂ and CN; and wherein R₁ and R₂ are each independently selected from OH, ORₐ, C(O)NR_{b}R_{c} and C(O)OR_{b}R_{c}; with the proviso that when R₁ is OH the compound of formula (I) is not menthol; and when the double bond is present, R₂ is absent;
wherein Rₐ is an alkyl group, an alkenyl group, a C(O)R_{f} group, or a C(O)-alkyl-C(O)R_{f} group wherein the alkyl groups and alkenyl groups are optionally substituted by one or more substituents selected from OH, O-alkyl, NH₂, NH-alkyl, N-(alkyl)₂, NO₂ and CN; and wherein R_{f} is an alkyl group, an alkenyl group, OH, O-alkyl, NH₂, NH-alkyl or N-(alkyl)₂, wherein the alkyl groups and alkenyl groups are optionally substituted by one or more substituents selected from OH, O-alkyl, NH₂, NH-alkyl, N-(alkyl)₂, NO₂ and CN;
wherein R_{b} and R_{c} are each independently hydrogen, an alkyl group, an alkenyl group, an aryl group, an aralkyl group, a heteroaryl group, or a heteroaralkyl group, wherein the alkyl groups, alkenyl groups, aryl groups and heteroaryl groups are optionally substituted by one or more substituents selected from OH, O-alkyl, NH₂, NH-alkyl, N-(alkyl)₂, NO₂, CN and C(O)R_{f}.
